(19) United States
(12) Patent Application Publication
(10) Pub. No.: US 2024/0309075 A1
(43) Pub. Date: Sep. 19, 2024

(54) MODULATION OF ANTIBODY FUNCTION VIA SIALIC ACID MODIFICATION

(71) Applicant: Children's Hospital Medical Center, Cincinnati, OH (US)

(72) Inventors: Sing Sing Way, Cincinnati, OH (US); John Erickson, Cincinnati, OH (US)

(21) Appl. No.: 18/279,250

(22) PCT Filed: Mar. 4, 2022
(86) PCT No.: PCT/US2022/018847

§ 371 Date: Aug. 29, 2023

Related U.S. Application Data
(60) Provisional application No. 63/234,811, filed on Aug. 19, 2021.
(60) Provisional application No. 63/156,403, filed on Mar. 4, 2021.

(51) Int. Cl. C07K 16/12; A61K 39/00

(52) U.S. Cl. CPC C07K 16/1296; A61K 2039/505; C07K 2317/41; C07K 2317/54; C07K 2317/71

(57) ABSTRACT

Disclosed are methods of modulating antibody function, in particular, via modifying one or more terminal sialic acid residues of an antibody. In certain aspects, the antibody may be an IgG antibody, wherein one or more terminal sialic acid residues is modified to modulate the antibody function. Further disclosed are methods of using such modified antibodies, and compositions comprising modified antibodies.

### CROSS-REFERENCE TO RELATED APPLICATIONS

This application claims priority to and benefit of U.S. Provisional Application Ser. No. 63/156,403, entitled ANTIBODIES AGAINST INTRACELLULAR ANTIGEN TARGETS, filed Mar. 4, 2021, and U.S. Provisional Application Ser. No. 63/234,811, entitled ANTIBODIES AGAINST INTRACELLULAR ANTIGEN TARGETS, filed Aug. 19, 2021. The contents of each are incorporated in their entirety for all purposes.

### STATEMENT REGARDING FEDERALLY-SPONSORED RESEARCH

This invention was made with government support under AI145184 and AI131080 awarded by the National Institutes of Health. The government has certain rights in the invention.

### REFERENCE TO SEQUENCE LISTING

A Sequence Listing submitted as an ASCII text file via EFS-Web is hereby incorporated by reference in accordance with 35 U.S.C. § 1.52e. The name of the ASCII text file for the Sequence Listing is CHMC_0753573_ST25.txt, the date of creation of the ASCII text file is Mar. 4, 2022, and the size of the ASCII text file is 4.0 KB.

### BACKGROUND

Antibodies, also known as an immunoglobulins, are produced by the body's immune system in response to a perceived harmful or foreign substance. Such proteins may be used, for example, as therapeutics and research tools. For example, monoclonal antibodies such as rituximab, a chimeric anti-CD20 IgG1 approved for non-Hodgkin's lymphoma, is an antibody that may be used for the therapeutic treatment of cancer. Further, antibodies may be useful as research tools, the specificity of antibodies serving an unique and useful role that may be leveraged as a detection tool. The use of antibodies as both a research tool and as a therapeutic tool may be improved upon, in particular, the immunomodulatory properties of antibodies may be altered in ways that improve the intended use of such antibodies. The instant disclosure seeks to address one or more of the aforementioned needs in the art.

### BRIEF SUMMARY

Disclosed are methods of modulating antibody function, in particular, via modifying one or more terminal sialic acid residues of an antibody. In certain aspects, the antibody may be an IgG antibody, wherein one or more terminal sialic acid residues is modified to modulate the antibody function. Further disclosed are methods of using such modified antibodies, and compositions comprising modified antibodies.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Maternal B cells are required for vertically transferred immunity against Lm infection. Female WT, μMT−/− or CD8−/− mice were primed with attenuated ΔActA Lm, mated with WT males 1 week later, and then 3 d old neonates were infected i.p. with Lm. Bacterial burdens were quantified at 72 h p.i. in the spleen or liver (a) and survival was monitored for 2 weeks (b). (c) WT females were primed with ΔActA Lm or virulent Lm, allowed to recover for 3 weeks before mating, and then neonates were infected with virulent Lm and bacterial burdens were quantified. (d) CD8−/− females were primed with Lm, mated, and then infected with Lm when 3 weeks post-partum. Naïve or primed virgin CD8−/− females were used as controls. (e) Females were primed with Lm, mated, and then 8 week old adult offspring were infected with Lm. Each dot represents the data from an individual mouse and graphs show data combined from 2-3 independent experiments each showing similar results. p-values between key groups are shown in each panel. Dotted lines indicate limit of detection.

FIG. 2. Pregnancy-enabled protection by anti-Lm antibodies requires maternal FcγR expression. (a) Immune sera from virgin (vSera) or pregnant mice at late gestation through early post partum (pSera) were transferred i.p. to 3 d old neonates. (b) pSera obtained at defined times during pregnancy or 5 weeks post-partum were collected and passively transferred to 3 d old pups. (c) Neonates were cross-fostered within 12 hours of birth with a naïve or primed WT female. (d) vSera were transferred to WT, μMT−/− or FcRγ−/− naïve postpartum females on days P0 and P3 that had been mated with WT males. For all experiments, pups were infected the day after sera transfer and organs were harvested 72 h post-infection. Each dot represents the data from an individual mouse and graphs show data combined from 2-3 independent experiments each showing similar results. p-values between key groups are shown in each panel. Dotted lines indicate limit of detection.

FIG. 3. N-glycan terminal sialic acid is required for IgG-mediated protection against Lm infection. (a) Schematic showing potential N-linked glycosylation on IgG Fc and Fab regions. (b) vSera was transferred to WT naïve postpartum females at P3 along with mock or i.v. injection of EndoS enzyme. (c) pIgG was treated in vitro with EndoS to remove N-glycans. IgG from naïve sera (nIgG) was used as a control. (d,e) Glycoengineering strategy to generate pIgG with defined glycan structures. Neuraminidase cleaves α2-3,6,8 linked sialic acid, while ST6Gal1 adds α2,6 linked sialic acid. CMP-Neu5Ac was used as the substrate. (f,g) Glycoengineering strategy to generate vIgG with defined glycan structures. For all experiments, IgG preparations were transferred to 3 d old neonates that were infected with Lm the following day. Bacteria were quantified in the spleen and liver 72 h post-infection. Each dot represents the data from an individual mouse and graphs show data combined from three independent experiments each showing similar results. p-values between key groups are shown in each panel. Dotted lines indicate limit of detection.

FIG. 4. Pregnancy deacetylates IgG sialic acid enabling antibody-mediated protection against Lm via CD22 and SIGN-R1. (a-b) Lm-specific IgG from virgin and pregnant mice was analyzed via mass spectroscopy. Mass peak area for Neu5Gc,Ac normalized to Neu5Gc content is shown for 2 representative glycopeptides (a). Percentage of Neu5Gc,Ac versus total sialic acid content for all 4 identified Fab glycopeptides. Average glycopeptide m/z based on oxonium ion filtering from 3 independent LC-MS runs (b). (c) CCA lectin was used to detect 9-O acetylated sialic acid on LLO-specific IgG (0.1 mg/mL final concentration) from virgin or pregnant/postpartum females. Symbols show relative light units for individual mice. (d) Siae gene expression was quantified in spleens of virgin and late gestation (E18-20) pregnant mice or in PBMCs from non-pregnant (NonPreg) versus pregnant humans. (e) vIgG was treated with SIAE and then transferred to 3 d old neonates. Naïve IgG (nIgG) and mock-treated vIgG were used as controls. (f) nIgG or pIgG were treated with pepsin to generate F(ab′)2 fragments and transferred to 3 d old neonates. (g) Neonatal mice were injected i.p. with naïve or pregnant Lm immune sera (Preg) in addition to αSIGN-R1 or αCD22 blocking monoclonal antibodies (mAb) or appropriate isotype control. Neonates were infected the day after IgG, F(ab′)2, or sera transfers and bacteria were quantified 72 h post-infection. Each dot represents the data from an individual mouse and graphs show data combined from three independent experiments each showing similar results. p-values between key groups are shown in each panel. Dotted lines indicate limit of detection.

FIG. 5. Additional data for FIG. 1. (a) WT females were primed with ΔActA Lm and then 3 d old neonates were infected with Candida albicans. Fungi were recovered from the spleen or liver 48 hours post-infection. (d) WT, B cell deficient (μMT−/−) or CD8+ T cell deficient (CD8−/−) mice (Naïve) or those that had been primed with ΔActA Lm four weeks prior (Prime) were challenged i.v. with virulent Lm. Bacteria burdens were quantified in the spleen 72 hours p.i. (c,d) CD8−/− females were primed with Lm, mated, and then infected when 3-4 weeks post-partum with virulent Lm. Naïve or primed virgin CD8−/− females were used as controls (c). Anti-Lm IgG was quantified prior to infection (d). Each dot represents the data from an individual mouse and graphs show data combined from 3 independent experiments each showing similar results. p-values between key groups are shown in each panel. Dotted lines indicate limit of detection.

FIG. 6. Vertically transferred antibody titers correlate with protection against Lm infection. (a) Serum anti-Lm titers were quantified from neonates born to ΔActA Lm primed WT or μMT−/−dams. (b) Serum anti-Lm titers were quantified from neonates born to ΔActA Lm primed or virulent Lm infected dams. (c-e) WT females were primed once or twice with ΔActA Lm, mated, and then 3 d old neonates were infected with virulent Lm. Bacterial burdens were quantified in the spleen and liver 72 hours post-infection. (c). OD450 for given serum dilutions for Lm-specific IgG from the blood of neonates (d) and EC50 were calculated (e). Each dot represents the data from an individual mouse and graphs show data combined from 2-3 independent experiments each showing similar results. p-values between key groups are shown in each panel. Dotted lines indicate limit of detection.

FIG. 7. Additional data for FIG. 2. (a-b) Immune sera from virgin (vSera) or pregnant mice at late gestation through early post-partum (pSera) were transferred i.p. to 3 d old neonates (a). EC50s were calculated for Lm-specific IgG from neonatal sera (b). (c-d) Neonates were cross-fostered within 12 hours of birth with a naïve or primed WT female and then infected with Lm (c). Serum anti-LLO antibodies were quantified from infected neonates (d). (e-f) vSera were transferred to WT, MT−/−, or FcR−/− naïve postpartum females on days P0 and P3 that had been mated with WT males and then WT or heterozygous pups were infected with Lm the following day (e). Lm-specific IgG was quantified in the sera of infected neonates (f). Each dot represents the data from an individual mouse and graphs show data combined from 2-3 independent experiments each showing similar results. p-values between key groups are shown in each panel. Dotted lines indicate limit of detection

FIG. 8. Pregnancy does not alter the quantity of Lm-specific IgG, Ab isotypes, IgG subclasses or lectin staining profiles. Female mice were primed with ΔActA Lm. IgG was purified from late gestation or early postpartum females along with time-matched IgG from virgin controls. (a) IgG titers were calculated for UV-inactivated Lm or recombinant LLO protein. (b) OD450 for sera from primed virgin or pregnant mice for the given Ab isotypes or subclasses. All sera diluted 1:100. Background subtracted from staining using naïve sera. (c) Lectins with defined carbohydrate specificity were used to determine the presence or absence of glycans purified IgG (0.1 mg/mL final concentration) binding to LLO toxin from individual virgin or late gestation/early post-partum female mice. Each dot represents the data from an individual mouse and graphs show data representing 1 of 2 independent experiments each showing similar results. p-values between key groups are shown in each panel. Dotted lines indicate limit of detection.

FIG. 9. Additional data for FIG. 7, (a,b) vSera were transferred to WT naïve postpartum females at P3 along with mock or i.v. injection of 10 g EndoS enzyme (a). Lm-specific IgG was quantified from the blood of infected neonates (b). (c) Lm-specific IgG was quantified from the blood of infected neonates that were transferred mock-treated or EndoS-treated pIgG. (d) Lectin staining to confirm the success of enzymatic reactions from FIG. 3, d, e. Decrease in SNA signal indicates removal of sialic acid, whereas an increase in ECA signal reveals uncovering of galactose by sialic acid removal. (e) Lm-specific IgG was quantified from the blood of infected neonates from FIG. 7, d, e. (f) Lectin staining to confirm the success of enzymatic reactions from FIG. 7, f, g. (g) Lm-specific IgG was quantified from the blood of infected neonates from FIG. 7, f, g. Each dot represents the data from an individual mouse and graphs show data combined from 2-3 independent experiments each showing similar results. p-values between key groups are shown in each panel. Dotted lines indicate limit of detection

FIG. 10. Oxonium ion searches for sialic acid modifications. (a) Schematic of process to isolate Lm-specific IgG from Lm-primed virgin or pregnant mice. Anti-Lm IgG was then subjected to trypsin or chymotrypsin digestion and LC-MS/MS analysis. (b) Sialic acid is modified from acetylneuraminic acid (Neu5Ac) to glycolylneuraminic acid (NeuGc) by the enzyme CMAH. A missense mutation renders CMAH inactive in humans, while mice retain its function, so murine IgG contains only Neu5Gc. Neu5Gc can be acetylated to make Neu5Gc,9Ac by the putative acetyl-transferase CASD1 and then deacetylated by sialic acid acetyl-esterase (SIAE). The addition of 9-O-acetylation is highlighted by a red circle. (c) Oxonium ion m/z used to search for sialic acid variants. Diagnostic ions were filtered through the MS/MS spectra to select glycopeptides containing acetylated sialic acid (Neu5Gc,Ac). Example spectra shown for virgin Lm-specific IgG. (d) MS/MS fragmentation for the low mass region (150-500) demonstrating sialic acid variants. All presented m/z are z=1. (e) Extracted ion chromatograph showing m/z corresponding to Neu5Gc,Ac and m/z of the same glycopeptides containing Neu5Gc. Examples of two individual glycopeptides are shown. Analyses were performed in triplicate.

FIG. 11. SIAE enzymatic treatment of Lm-specific IgG from pregnant mice does not enhance protective capability. (a) vIgG was treated with SIAE and then transferred to neonates. Lm-specific IgG was quantified from the blood of infected neonates. (b, c) pIgG was treated with SIAE and then transferred to neonates and bacterial burdens (b) and Lm-specific IgG was quantified in the blood 72 h post-infection. Each dot represents the data from an individual mouse and graphs show data combined from 2-3 independent experiments each showing similar results. p-values between key groups are shown in each panel. Dotted lines indicate limit of detection.

FIG. 12. Acetylated sialic acid localizes to the IgG variable region. (a) Lm-specific IgG from virgin or pregnant mice was subjected to trypsin or chymotrypsin digestion and then LC-MS/MS analysis. Example of MS/MS Fragmentation from the conserved N-linked glycosylation site on the Fc region of IgG2b (glycopeptide EDYNSTLR (SEQ ID NO: 1). (b) Extracted ion chromatograph of IgG2b peptide EDYNSTIR (SEQ ID NO: 1) with the observed sialic acid (Neu5Gc) glycoforms and absence of acetylated sialic acid (Neu5Gc,Ac). (c) % of IgG2b conserved region N-glycans with given glycoforms. NG=no glycosylation, G0=agalactosylated, G1/2=mono- or digalactosylated, G2S1=monosialylated, G2S2=disialylated. (d) Example of MS/MS fragmentation (glycopeptide N₁E₂S₁L₁Y₁T₁A₂I₁K₁) (SEQ ID NO: 2) demonstrating presence of Neu5Gc,Ac. (e) Neu5Gc,Ac localizes to 4 novel peptides in the Fab region, 2 of which were in sufficient abundance to determine the amino acid composition and N-linked glycoforms.

FIG. 13. (a-c) pIgG was treated with pepsin to remove the Fc region (a). F(ab′)2 were compared to parent pIgG for Lm binding and presence of H+L chains, IgG1 and IgG2b (b). pIgG-derived F(ab′)2 was transferred to 3 d old neonates. Lm-specific F(ab′)2 was quantified from the blood of infected neonates with IgG H+L secondary antibody (c). (d-f) Neonatal mice were injected with naïve or pregnant Lm immune sera in addition to CD16/32 (d,e), SIGN-R1 or CD22 (f) blocking monoclonal antibodies (mAb) or appropriate isotype control. Lm-specific IgG was quantified from the blood of infected neonates (e,f). Neonates were infected the day after F(ab′)₂ or sera transfers and bacteria were quantified 72 h post-infection. *** indicates p<0.0005 for Preg samples compared to Naive samples. Each dot represents the data from an individual mouse and graphs show data combined from three independent experiments each showing similar results. p-values between key groups are shown in each panel. Dotted lines indicate limit of detection.

FIG. 14. Complement is dispensable in neonates for antibody-mediated protection against Lm. 3 d old WT, C1q−/− (a) or C3−/− (b) neonates were passively transferred naïve sera or pSera and then infected with Lm the following day. Bacterial burdens were quantified in the spleen and liver 72 hours post-infection. Each dot represents the data from an individual mouse and graphs show data combined from two independent experiments each showing similar results. p-values between key groups are shown in each panel. Dotted lines indicate limit of detection.

FIG. 15. Glycoforms on conserved IgG2b FcN-linked glycosylation site from virgin or pregnant Lm-specific IgG.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

In one aspect, a method of modulating antibody function is disclosed. In this aspect, the method may comprise modifying a terminal sialic acid residue of an antibody. For example, the modification may comprise removing an existing sialic acid, and replacing the sialic acid residue with a sialic acid variant of interest. In one aspect, the method may comprise treating an antibody with a neuraminidase, followed by treatment with ST6Gal1 and the sialic acid variant of interest. In one aspect, the term “sialic acid” refers to an N-substituted or O-substituted derivative of neuraminic acid (a monosaccharide having a 9 carbon skeleton). One member of this family of neuraminic acid derivatives is N-acetylneuraminic acid (Neu5Ac or NANA). Other suitable sialic acids may include, for example, N-glycolylneuraminic acid (NGNA or Neu5Gc), N-acetyl group of Neu5Ac is hydroxylated, or 2-keto-3-deoxynonurosonic acid (KDN), or a 9-substituted sialic acid, (for example 9-O—C—C6 acyl Neu5Ac), or a 9-deoxy, (for example, 9-O-lactyl-Neu5Ac or 9-O-acetyl-Neu5Ac), 4-O-Acetyl-SA, 7,9-O-Acetyl-SA, 9-Fluoro-Neu5Ac and 9Azido-9-deoxy-Neu5Ac, and combinations thereof. The sialic acid may be one described in, for example, U.S. Pat. No. 8,632,773. In one aspect, the sialic acid may be acetylneuraminic acid (Neu5Ac).

In one aspect, the modification introduced to the antibody using the described methods may be measured via lectin staining. Lectins are derived from plants or animals and bind to carbohydrates with very specific configurations. For sialic acid, SNA lectin staining may be used. SNA signal decreases with neuraminidase treatment (the sialic acid is removed) and then the signal would increase after ST6Gal1+Sialic acid substrate treatment. Mass spectroscopy may also be used to measure the presence of carbohydrates based on their known or predicted mass sizes.

In one aspect, the antibody function that may be modified may be one or more of a protective effect, a therapeutic effect, and an immune response to an antigen. In one aspect, the modulating of the antibody may comprise an increase or decrease in said antibody function. In one aspect, the modifying of the antibody may comprise removing a terminal sialic acid residue of said antibody, for example, one or more of adding a terminal sialic acid residue of said antibody, acetylation of said terminal sialic acid residue, de-acetylation of said terminal sialic acid residue. De-acetylation is carried out via contact with sialic acid acetyl esterase (SIAE). Addition of a sialic acid may be carried out, in one aspect such contacting being carried out following the de-acetylation, via contacting an antibody with ST6 beta-galactosidase alpha-2,6-sialyltransferase 1 (ST6Gal1) sufficient to add at least one α2,6-linked sialic acid to a galactose residue of said antibody. In one aspect, the method may comprise the step of contacting the antibody with an endoglycosidase, contacting the antibody with neuraminidase digestion until said antibody is desialylated, contacting the antibody with neuraminidase followed by treatment with a sialyltransferase, contacting the antibody with neuraminidase followed by treatment with a sialyltransferase selected from ST3Gal III (α-(2,3) sialyltransferase and α-(2,6) sialyltransferase, contacting the antibody with one or more of ST6 beta-galactosidase alpha-2,6-sialyltransferase 1 (ST6Gal1) enzyme and a sialic acid, or combinations thereof. In one aspect, a step that removes a sialic acid precedes a step which adds a sialic acid. In one aspect, a step that modifies a sialic acid follows one or more steps that remove and/or adds a sialic acid. In one aspect, a sialic acid derivative may be added to an IgG, said sialic acid being directly acetylated. For example, a ST6Ga11 plus 9-O-Acetyl-Neu5Ac may be used to add the acetylated variant.

In one aspect, the antibody that is modified may be an immunoglobulin G-type (IgG) antibody. In one aspect, the antibody may be an immunoglobulin G-type (IgG) antibody, for example, wherein the terminal sialic residue may be located on the heavy chain of the IgG antibody. In one aspect, the terminal sialic acid residue may be located on a variable region of a heavy chain of the IgG antibody. In one aspect, the terminal sialic acid residue may be located on the N-linked glycan in the IgG variable region of the antibody.

In one aspect, the method may comprise removing a 9-O-linked acetylation or 7-O-linked acetylation from a sialic acid on an immunoglobulin G-type (IgG) antibody, comprising contacting the IgG with a sialylate O-acetylesterase (SIAE). In this aspect, the sialic acid residue may be located on a variable region of the heavy chain of the IgG antibody.

In one aspect, a method comprising adding an acetylation to sialic acid on an immunoglobulin G-type (IgG) antibody is disclosed, in which the method may comprise contacting the IgG with a sialic acid O-acetyltransferase (SOAT). In one aspect,

In one aspect, a method of modulating an antibody-related immune response in an individual is disclosed. In this aspect, the method may comprise administering an antibody as described herein. In this aspect, the administration of the antibody may be in an amount and for a period of time sufficient to improve a disease outcome. For example, the outcome may be selected from one or more of reduction in pathogen burden and survival, disease-free survival, symptom improvement or survival, and combinations thereof.

In further aspects, disclosed are compositions which may comprise one or more of the aforementioned antibodies having one or more of the disclosed sialic acid modifications. Such composition may comprise one or more pharmaceutically acceptable excipients and/or diluents, for example, an administration form suitable for administration to an individual in need thereof, for example the diluent being a sterile and/or buffered saline.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Adaptive immune components are thought to exert nonoverlapping roles in antimicrobial host defense, with antibodies targeting pathogens in the extracellular environment and T cells eliminating infection inside cells^{1,2}. Reliance on antibodies for vertically transferred immunity from mothers to babies may explain neonatal susceptibility to intracellular infections³. Applicant found that pregnancy-induced post-translational antibody modification enables protection against the prototypical intracellular pathogen Listeria monocytogenes (Lm). Lm infection susceptibility was overturned in neonatal mice born to preconceptually primed mothers containing Lm-specific antibodies or upon passive transfer of antibodies from primed pregnant, but not virgin, mice. While maternal B cell antibody production was essential for vertically transferred immunity, B cells were dispensable for antibody acquisition of protective function mediated by deacetylation of terminal sialic acid residues on N-linked glycans located in the IgG variable region. Expression of the deacetylating enzyme sialic acid acetyl esterase (SIAE)⁴ increased in human and mouse pregnancy and SIAE-mediated deacetylation alone unleashed protective function of Lm-specific IgG through the sialic acid receptors CD22 and SIGN-R1^{5,6}. Consideration of the maternal-fetal dyad as a joined immunological unit unveils newfound protective roles for antibodies against intracellular infection and exquisitely fine-tuned adaptations to enhance host defense during pregnancy and early life.

Infection remains a leading cause of neonatal mortality^{7,8}. Antibodies broadly defend against infection but offer limited protection against pathogens residing inside cells, which are the primary targets of T cell-mediated immunity^{1,2}. This division of labor between adaptive immune components may explain why newborn babies that rely on vertically transferred maternal antibodies are especially susceptible to intracellular infections⁹. The intracellular niche is exploited by many pathogens that cause severe perinatal infection including the Gram-positive bacterium Lm^{9,10} which rapidly gains access to the cell cytoplasm after phagosome escape using the pore-forming toxin listerolysin O (LLO), thereafter spreading from the cytoplasm of one cell to another via ActA-mediated host cell actin polymerization^{11,12}.

Vertically transferred maternal immunity primed by natural infection¹³, vaccination¹⁴ or commensal colonization of mothers¹⁵ dictates the adaptive immune repertoire of neonates¹⁶. Inadequate acquisition of protective maternal antibodies in premature or formula-fed infants each result in increased infection risk, including sepsis, lower respiratory infections and diarrheal illnesses^{17,18}. However, since immune mediators that protect against Lm and other intracellular pathogens have primarily been defined after passive transfer into adult recipients¹⁹⁻²⁴, relevance to how vertically transferred immunity protects neonates remains uncertain. Shared susceptibility to Lm infection between human babies and neonatal mice was exploited to probe how changes unique to the maternal-fetal dyad control immunity against intracellular infection.

### Vertical Immunity Needs Maternal B Cells

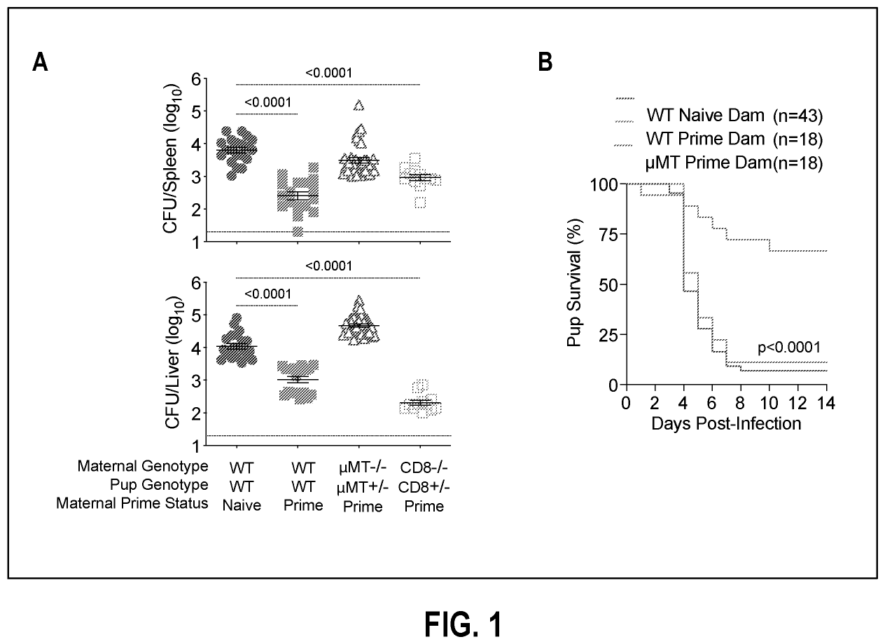

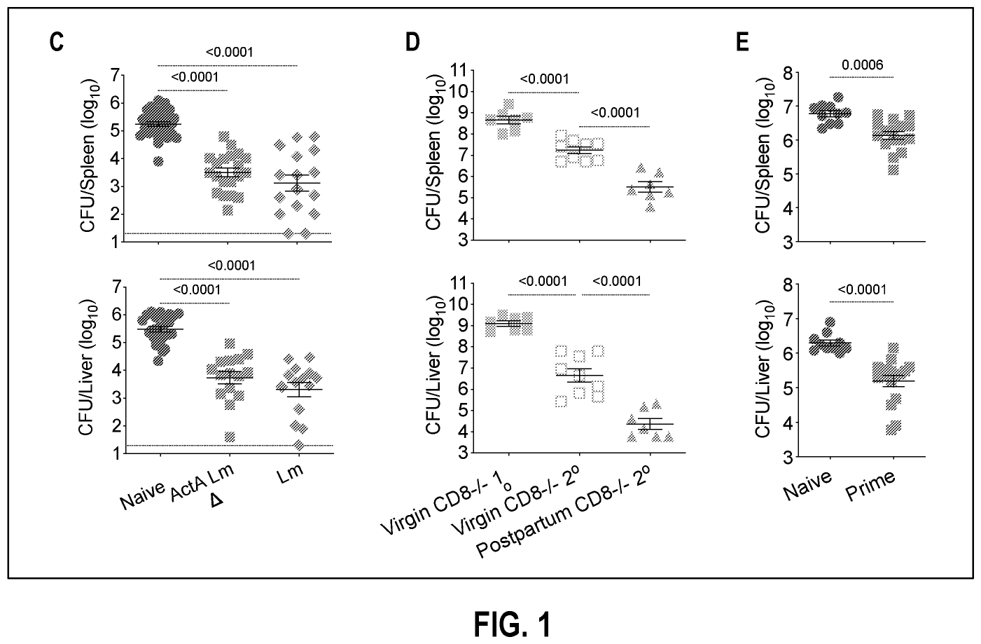

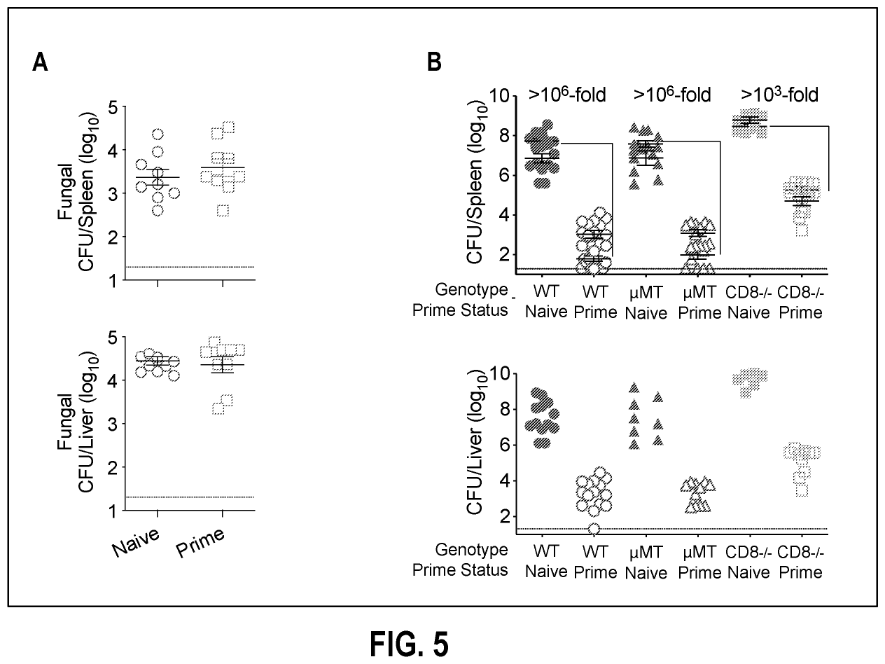

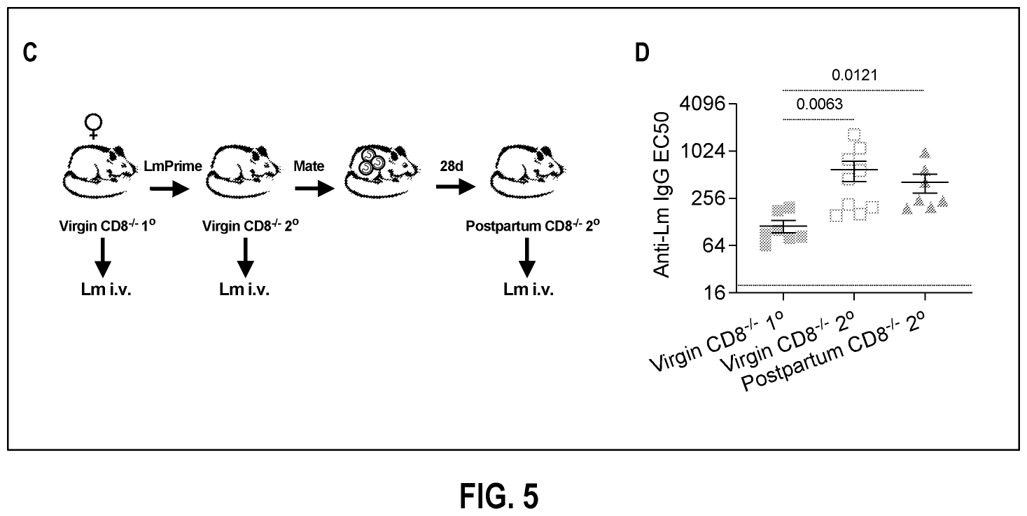

To investigate vertically transferred immunity against intracellular infection, susceptibility of neonatal mice born to preconceptually primed Lm immune mothers was evaluated. To eliminate the possibility of vertically transferred infection, attenuated ΔActA Lm, which is rapidly cleared but retains immunogenicity even in immunocompromised mice², was used to prime virgin female mice. Upon virulent Lm infection, neonatal mice born to primed mothers, compared with age-matched isogenic controls born to naïve mothers, contained reduced bacteria in the spleen and liver that paralleled significantly enhanced overall survival (FIG. 1, a, b). Analogous experiments evaluating pups born to B cell deficient (μMT−/−) or CD8+ T cell deficient mothers showed vertically transferred immunity against Lm infection was completely abolished in ΔActA Lm primed μMT−/− dams but persisted in primed CD8−/− mothers (FIG. 1, a, b). Use of WT males to sire pregnancy ensured these results did not reflect the absence of B or T cells in offspring. Vertically transferred protection was not restricted to attenuated ΔActA Lm priming, as pups born to dams with resolved preconceptual virulent Lm infection were similarly protected (FIG. 1, c) and occurred in a pathogen-specific manner since neonatal mice born to Lm-primed dams showed equal susceptibility to the unrelated fungal pathogen Candida albicans (FIG. 5, a).

Given these counterintuitive roles for humoral compared with cellular adaptive immune components in the context of vertically transferred anti-Lm immunity, the previously reported importance of CD8+ T cells and non-essential role of B cells for protection against Lm challenge in virgin adult mice was verified (FIG. 5, b). In contrast, significantly reduced Lm susceptibility after pregnancy in primed CD8−/− mice containing anti-Lm antibodies indicated that the importance of CD8+ T cells for anti-Lm immunity was bypassed by pregnancy (FIG. 1, d, FIG. 5, c, d). Vertically transferred protection was not limited only to neonatal infection, as significantly reduced susceptibility to virulent Lm challenge was found in 8-week-old adult mice born to Lm-primed mothers (FIG. 1, e). Thus, pregnancy is the dominant physiological feature that overrides the importance of CD8+ T cells, simultaneously transforming B cells into critical mediators of vertically transferred anti-Lm immunity.

### Pregnancy Enables Antibody Protection

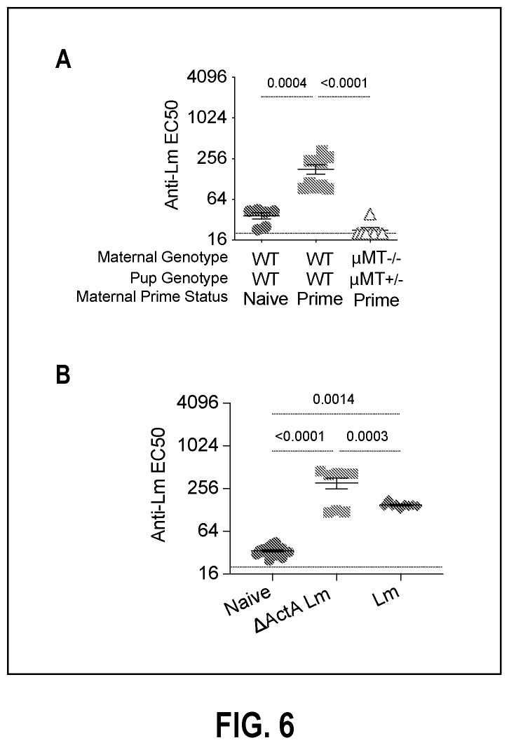

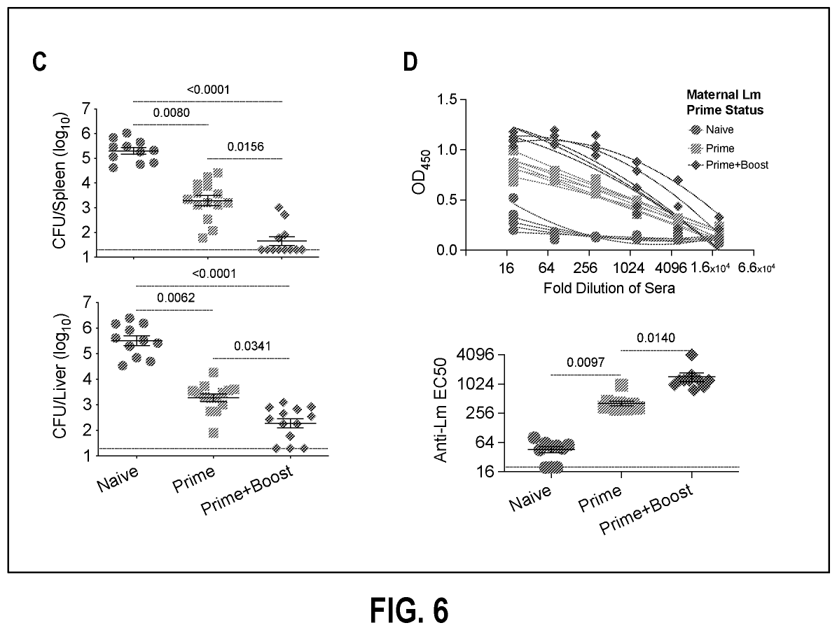

Maternal B cell-mediated vertically transferred protection against Lm infection was achieved despite the relatively low quantity of Lm-specific IgG primed by attenuated or virulent Lm infection compared with the expected background levels found in the highly susceptible pups born to primed μMT−/− dams (FIG. 1, a; FIG. 6, a, b). Susceptibility of neonatal mice to Lm infection was further reduced for pups possessing higher titer anti-Lm IgG born to mothers primed with a second dose of attenuated Lm (FIG. 6, c, d), highlighting the association between quantity of vertically transferred anti-Lm antibodies and neonatal protection from infection.

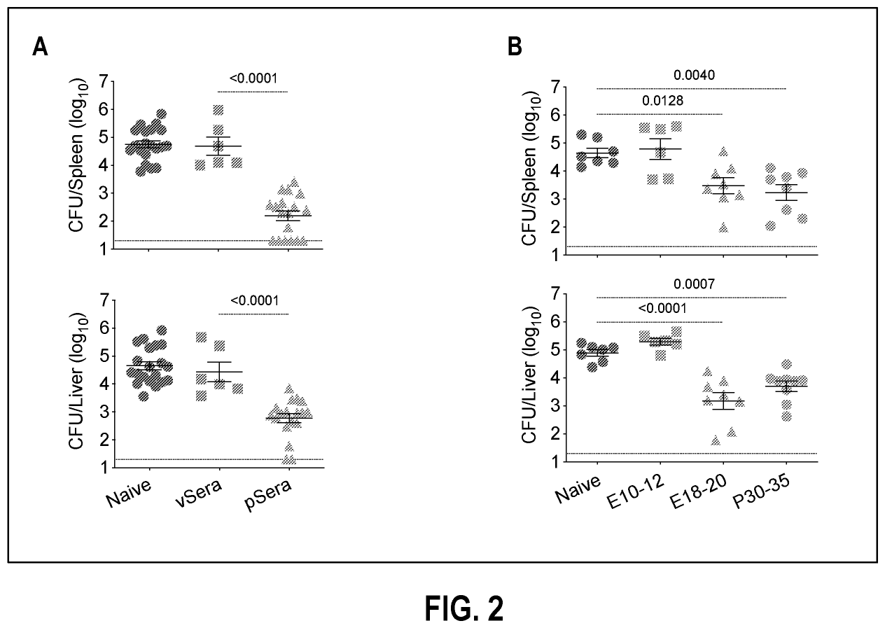

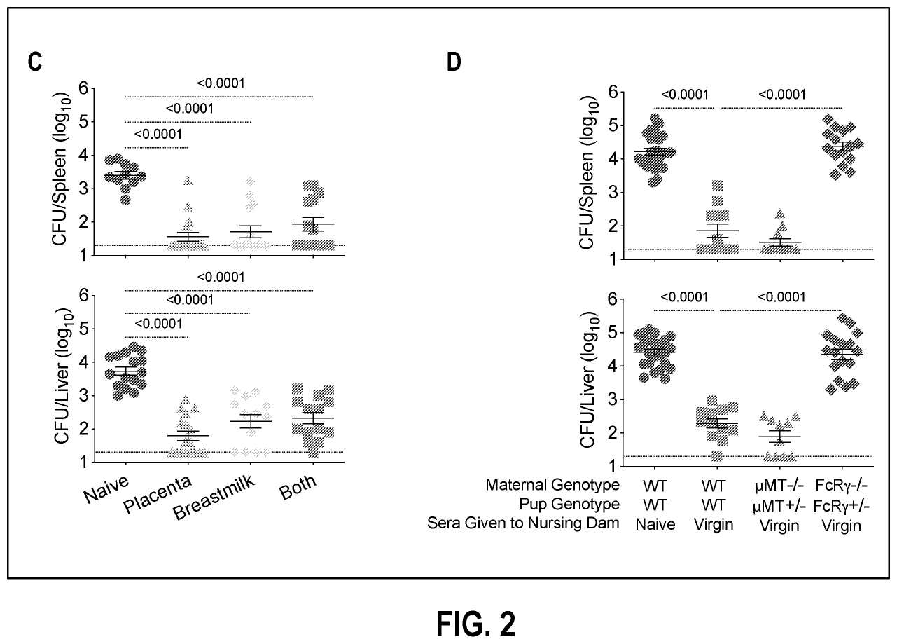

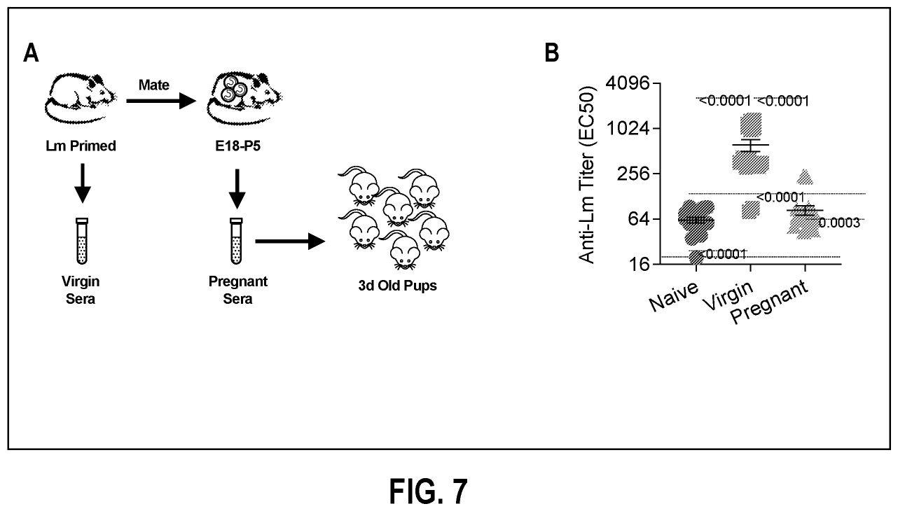

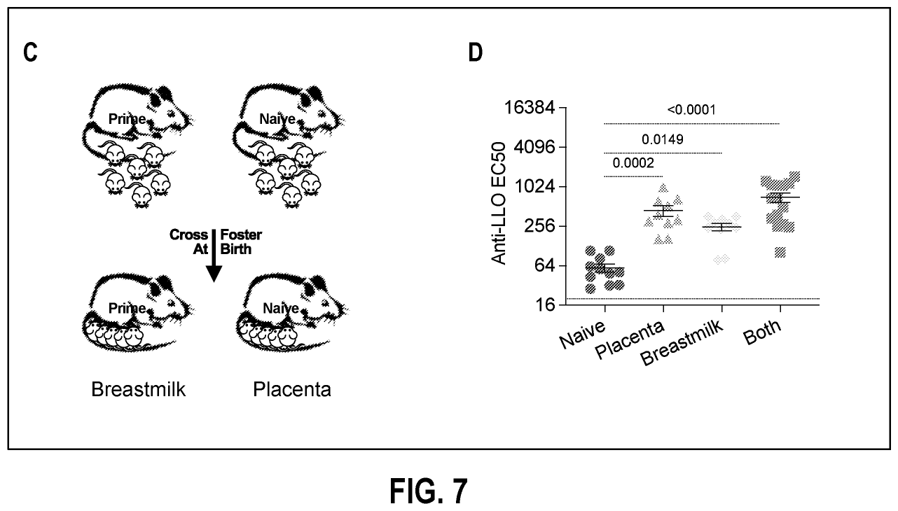

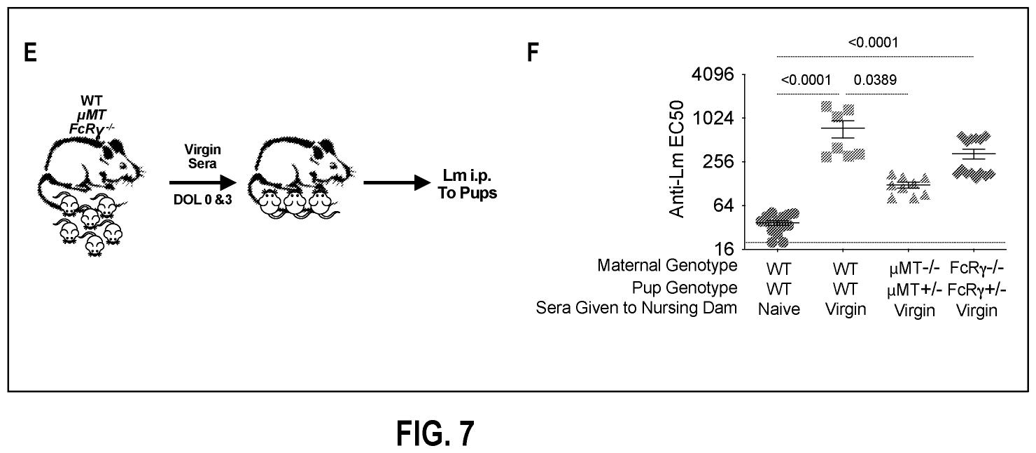

Passive transfer experiments were utilized to directly test antibody-mediated protection. Sera containing anti-Lm IgG from primed pregnant and postpartum dams (pSera) administered directly to neonates recapitulated vertically transferred protection, while sera from Lm-primed virgin mice (vSera) were non-protective despite higher anti-Lm antibody titer achieved in neonates (FIG. 2, a, FIG. 7, a, b). Maternal antibody acquisition of protective function occurred in late gestation and persisted for several weeks after parturition, as donor sera harvested from primed late gestation (E18-20) or postpartum (P30-35) mice were similarly protective, whereas sera harvested from primed mid-gestation (E10-12) donor mice were non-protective (FIG. 2, b).

Cross-fostering studies demonstrated robust transfer of Lm-specific IgG regardless of in utero or postnatal acquisition via breastfeeding, each leading to similarly reduced neonatal Lm susceptibility (FIG. 2, c, FIG. 7, c, d). Efficient breastmilk transfer of maternal antibodies was exploited to further investigate mechanistic details, including whether pregnancy enabled protection via de novo antibody production or post-translational modification of pre-existing antibodies. Remarkably, non-protective vSera indirectly transferred to pups through nursing dams gained the ability to protect neonates against infection (FIG. 2, d compared with FIG. 6, a; FIG. 7, e). Maternal antibody acquisition of protective function during pregnancy was dissociated from antibody production at the cellular level, as protection against Lm infection was also found in pups nursed by B cell-deficient (μMT−/−) dams passively transferred vSera. By contrast, maternal Fcγ receptors (FcγR), broadly required for antibody functions²⁶, were essential for protective conversion, since pups nursed by FcR γ-chain deficient (FcRγ−/−) dams passively transferred vSera remained susceptible to Lm infection. Importantly, neither the absence of maternal B cells nor FcγR impacted the transfer of antibodies to pups through breastfeeding as similar anti-Lm IgG titers were recovered from pups nursed by dams of each genotype (FIG. 7, f). Additionally, use of WT male mice to sire pregnancy in μMT−/− and FcRγ−/− female mice to generate phenotypically WT (μMT+/− and FcRγ+/−) offspring excluded the possibility that susceptibility differences reflect lack of B cells or FcγR in neonates. Thus, while maternal B cells are essential for producing antibodies that mediate vertically transferred immunity (FIG. 1), they are dispensable for pregnancy-induced acquisition of protective function, which instead relies on additional FcγR-bearing maternal leukocytes.

### Protective Antibodies Require Sialic Acid

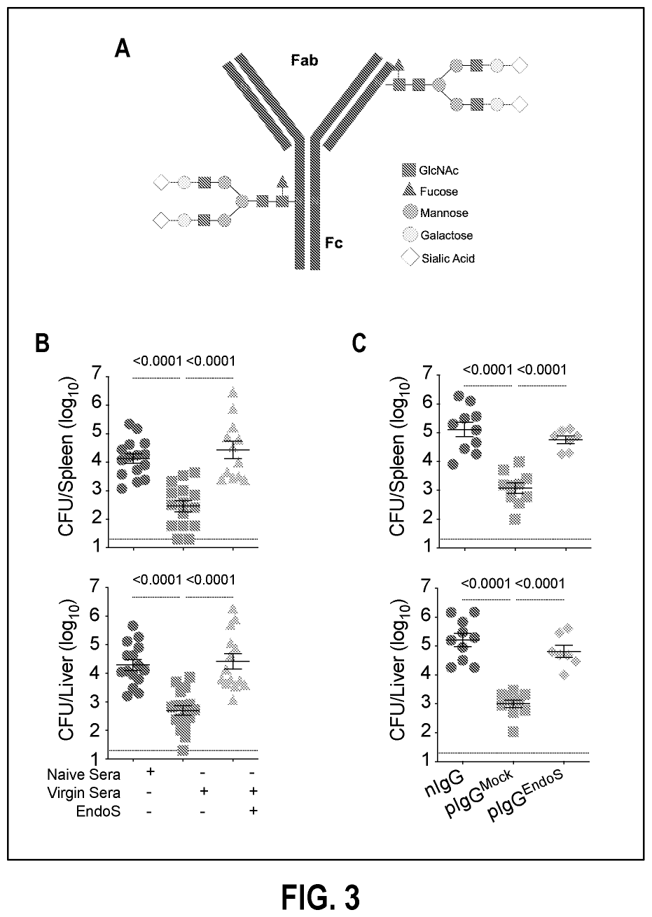

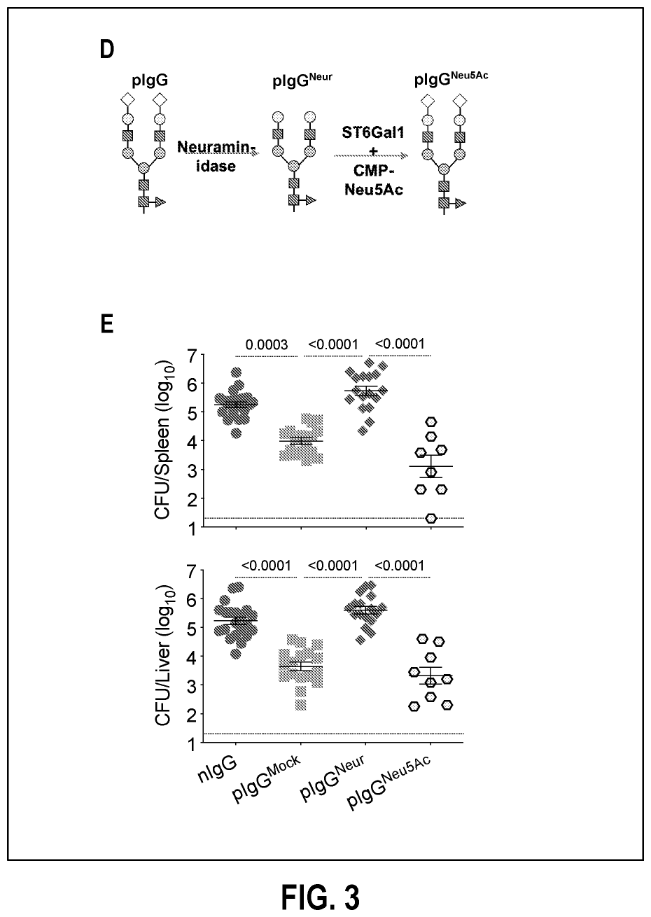

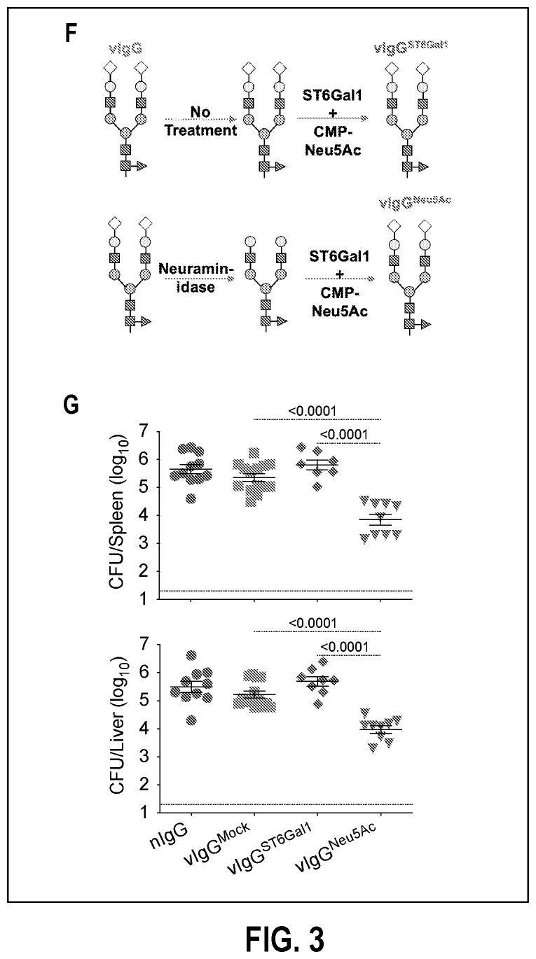

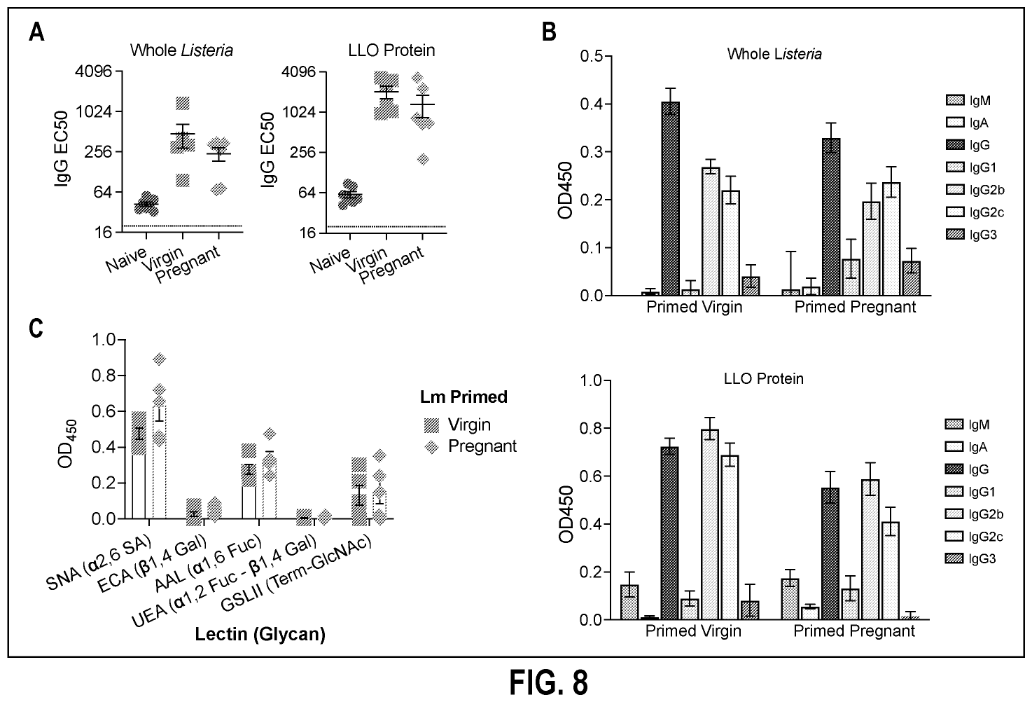

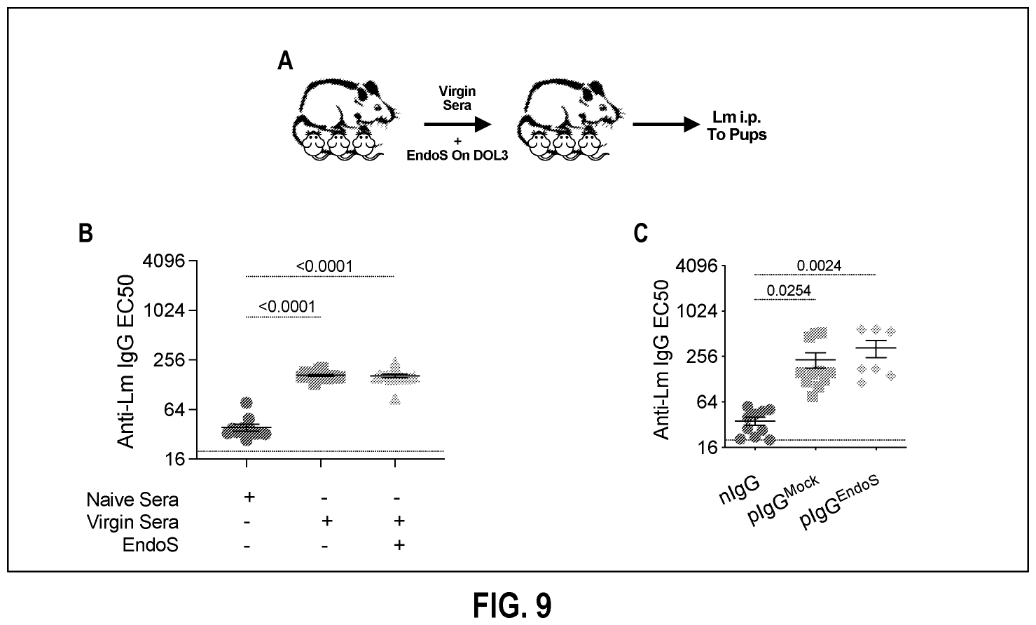

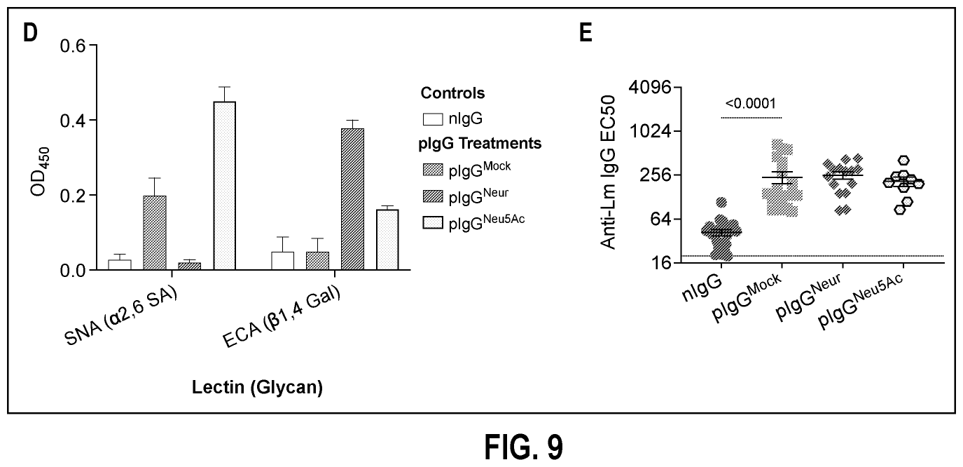

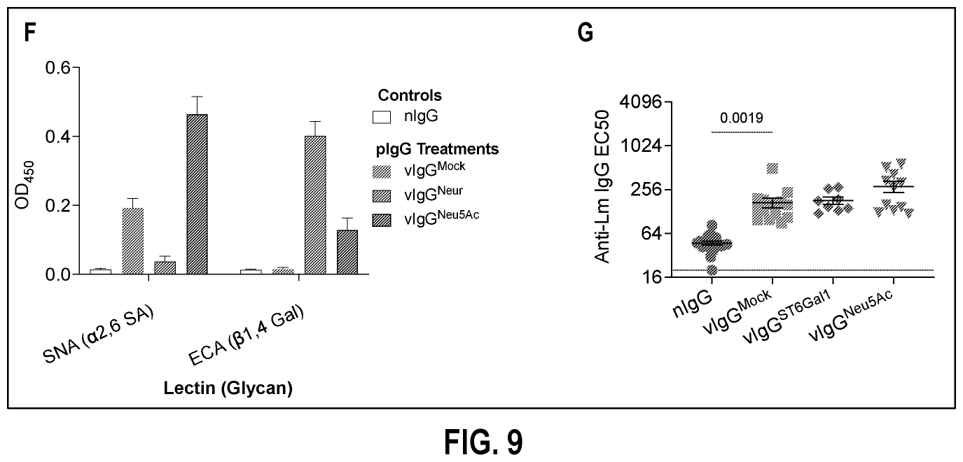

Anti-Lm antibodies recovered from virgin compared with pregnant/postpartum mice were further evaluated to determine the molecular features responsible for protection against intracellular infection. Pregnancy altered neither the overall titer nor levels of individual isotypes of serum antibodies with specificity to UV-inactivated Lm- or purified LLO toxin, with predominant accumulation of IgG2 (FIG. 8, a, b). Since post-translational antibody modification by N-linked glycosylation optimizes placental IgG transfer and protective function in neonates (FIG. 3, a)^{27,28}, the impact of endoglycosidase S (EndoS) selective cleavage of the N-linked glycan core²⁹ on anti-Lm IgG-mediated protection was assessed. EndoS coadministration with vSera to nursing dams overturned maternally-transferred protection against Lm infection (FIG. 3, b; FIG. 9 a). The protective properties of purified IgG from sera of pregnant mice containing anti-Lm antibodies (pIgG) were similarly eliminated after in vitro EndoS treatment without affecting neonatal antibody retention (FIG. 3, c; FIG. 9, b, c), confirming that antibody N-linked glycosylation is required for protection against intracellular infection.

Glycosylation profiles of anti-Lm IgG from virgin and pregnant mice were compared using a panel of lectins with defined-carbohydrate specificity. Despite a similar overall pattern of lectin staining, surprisingly high levels of α2,6-linked sialic acid were found (FIG. 8, c), which is generally thought to be the terminal step in antibody glycosylation and reduces IgG affinity for FcγR28. IgG N-glycan terminal sialic acid necessity was evaluated by neuraminidase digestion, showing sialic acid removal abolished pIgG-mediated protection against neonatal Lm infection (FIG. 3, d, e). Reciprocally, protective properties of neuraminidase-desialylated IgG were completely restored by ST6 beta-galactosidase alpha-2,6-sialyltransferase 1 (ST6Gal1) enzymatic treatment, which adds α2,6-linked sialic acid to galactose residues, plus the most common form of human sialic acid acetylneuraminic acid (Neu5Ac) as substrate (FIG. 3, e; FIG. 9, d, e).

Given similar levels of anti-Lm IgG sialylation in pregnant compared with virgin mice, and known molecular variations in N-glycan sialic acid³⁰, Applicant evaluated whether alterations in IgG N-glycan sialic acid were responsible for differences in protective function. Remarkably, replacing native sialic acid on IgG from Lm-primed virgin mice (vIgG) using neuraminidase followed by treatment with ST6Gal-1 plus Neu5Ac restored protective function (FIG. 3, f, g). By contrast, forcing more sialic acid onto vIgG using ST6Gal1 plus Neu5Ac without neuraminidase pre-treatment was ineffective, despite similar levels of antibody retention in neonates (FIG. 9, g). Thus, anti-Lm IgG N-glycan terminal sialylation may be essential, and sialic acid molecular variations between pIgG and vIgG is believed to be responsible for discordant protective function.

### Pregnancy Deacetylates IgG Sialic Acid

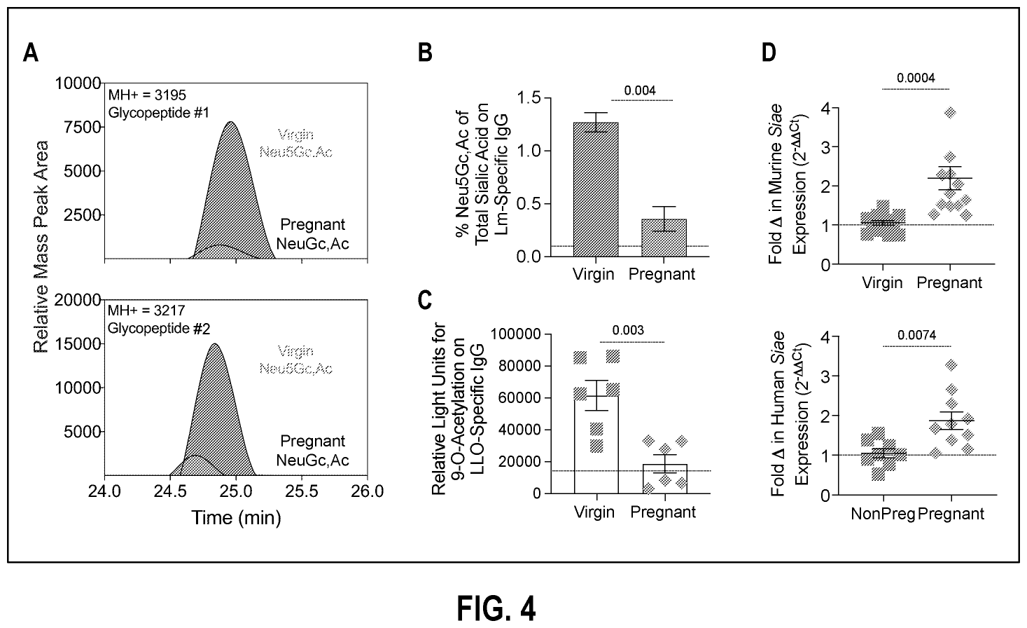

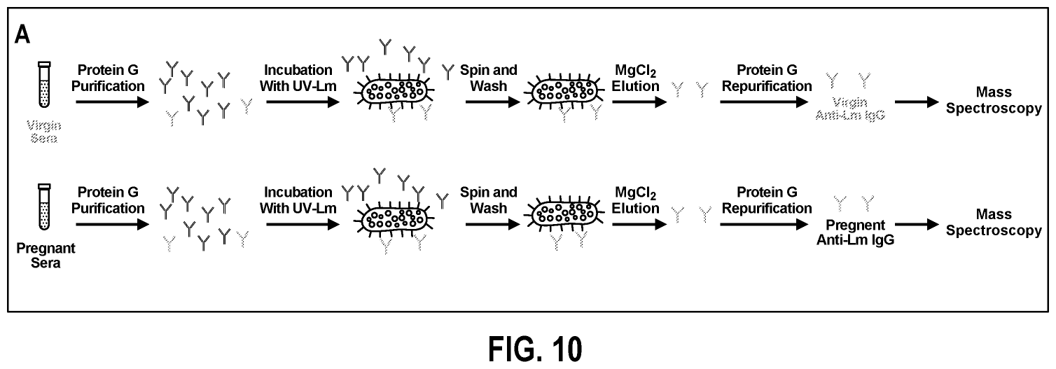

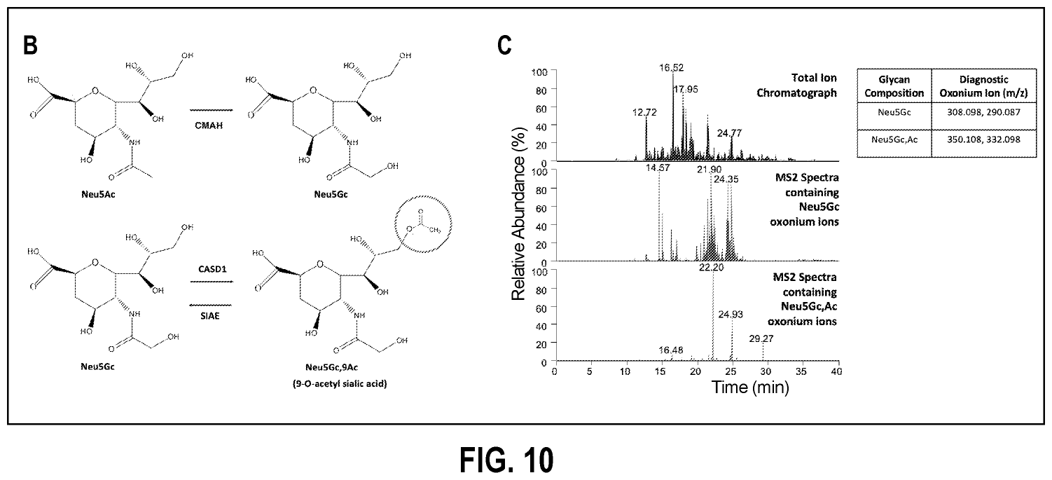

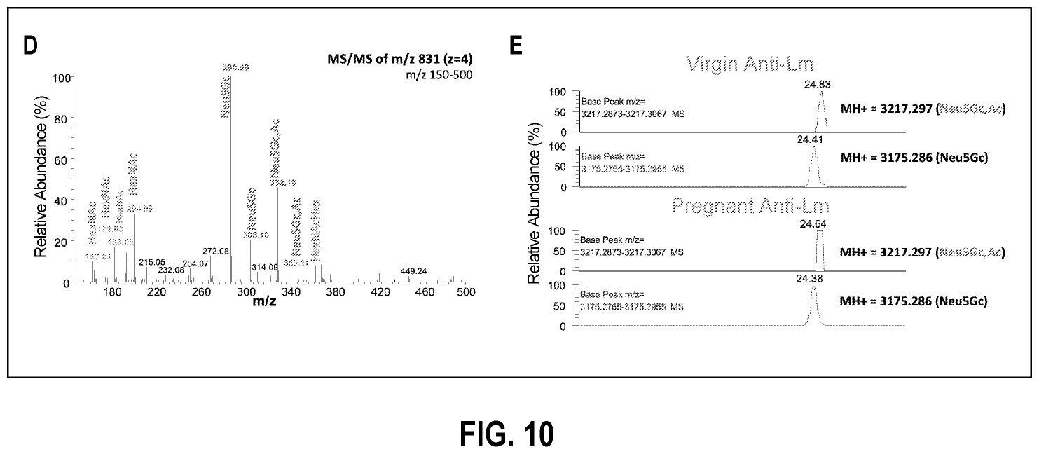

Mass spectroscopy analysis of protective pregnant and non-protective virgin Lm-specific IgG after trypsin and chymotrypsin digestions were performed to distinguish sialic acid modifications based on oxonium ion signatures³¹ (FIG. 10, a-c). Remarkably, acetylated murine sialic acid (O-acetyl-glycolylneuraminic acid, Neu5Gc,Ac), not previously described on IgG N-glycans, was greatly increased in virgin compared with pregnant Lm-specific IgG (FIG. 4, a, b; FIG. 10, d). The observed 42 Dalton mass increase over Neu5Gc exactly corresponds to addition of a single acetyl group, excluding release of acetylation from other biomolecules (FIG. 10, e). Staining using the Cancer antennarius lectin with specificity for 9-O-acetylated sialic acid32 confirmed similarly increased acetylation on terminal sialic acid residues of LLO-specific IgG in virgin compared with pregnant mice (FIG. 4, c).

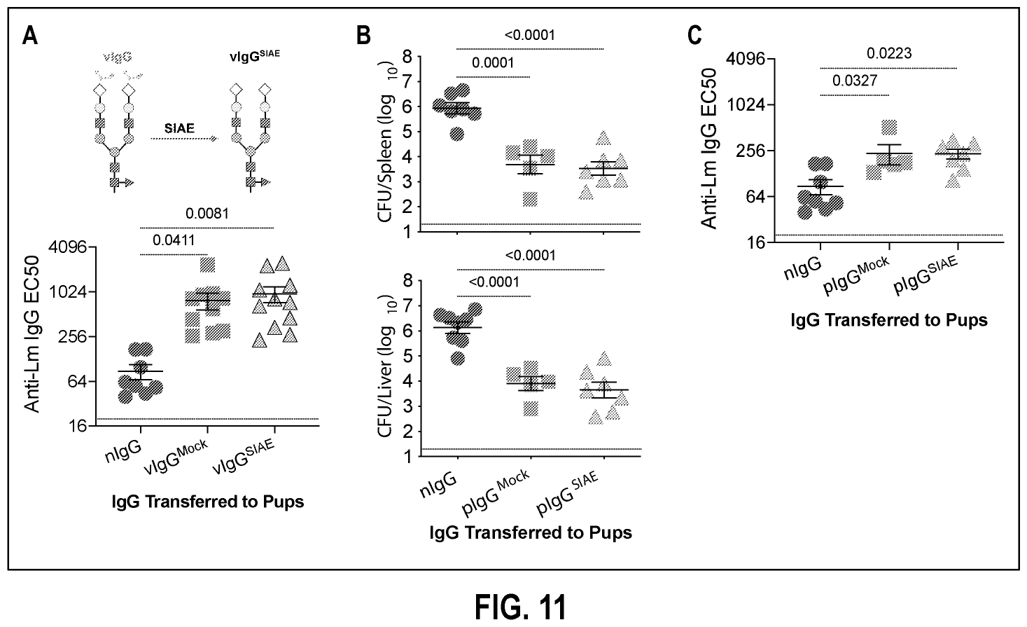

Sialic acid deacetylation is mediated by sialic acid acetyl esterase (SIAE)33 and polymorphisms in this enzyme are linked with autoimmunity and pregnancy complications^{34,3}. Applicant found SIAE expression also significantly increased during pregnancy in humans and mice (FIG. 4, d), suggesting dynamic regulation of N-glycan acetylation during pregnancy. To investigate whether SIAE-mediated sialic acid deacetylation is a key molecular determinant enabling Lm-specific antibody protective function, the impact of enzymatic deacetylation on vIgG protective function was evaluated. Applicant found sharply reduced bacterial burdens in pups adoptively transferred SIAE-compared with mock-treated vIgG (FIG. 4, e; FIG. 11, a), whereas the protective potency of pIgG was not further enhanced by SIAE treatment (FIG. 11, b, c). Thus, sialic acid deacetylation during pregnancy efficiently unleashes IgG protective function against intracellular infection.

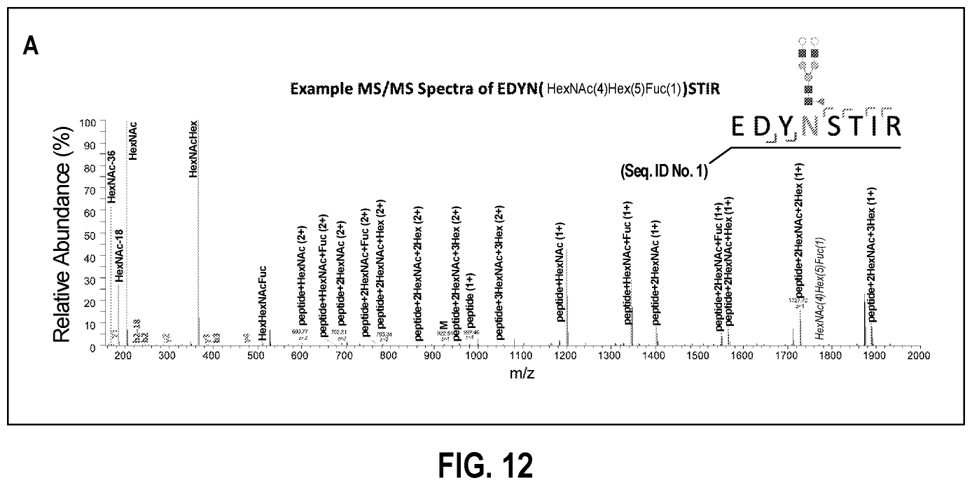

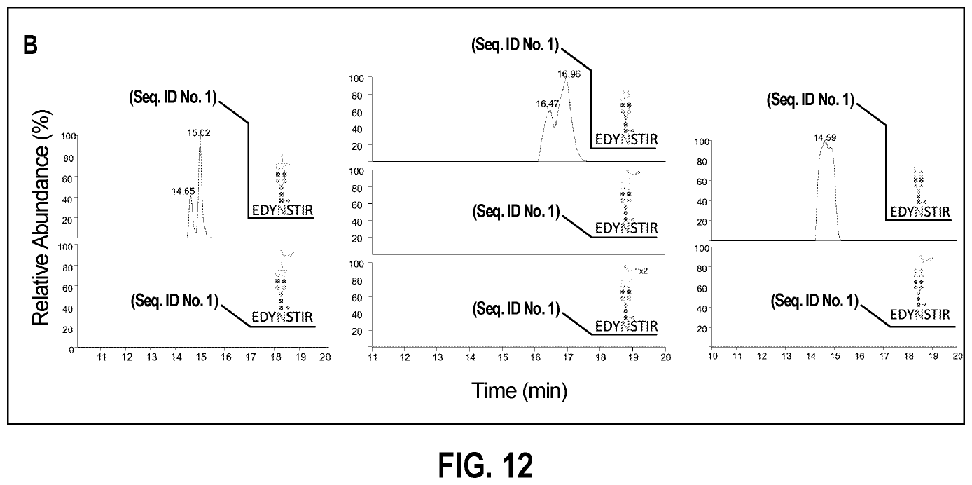

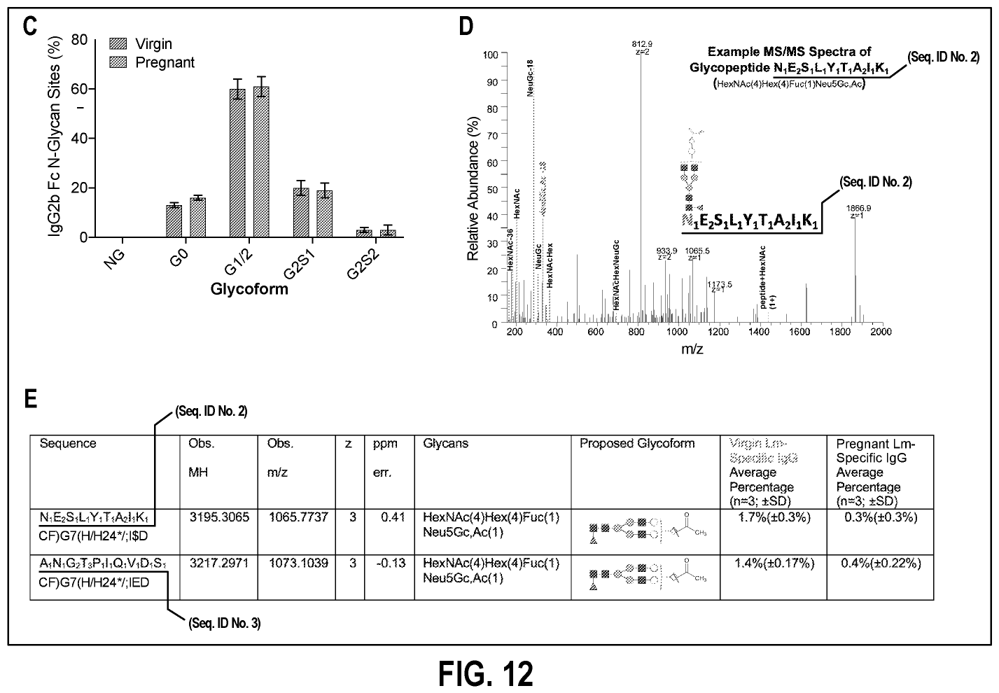

Glycopeptide mapping surprisingly revealed the complete absence of Neu5Gc,Ac from the conserved Fc region of Lm-specific IgG in both virgin and pregnant mice, with only 20-25% of IgG2 Fc sites containing any Neu5Gc (FIG. 12, a-c and Tables 1,2) and IgG1/IgG3 in very low abundance with minimal Fc sialylation (data not shown). Instead, Neu5Gc,Ac on biantennary N-glycans was only identified in sufficient abundance for unique glycopeptides that did not correspond to any known murine proteins (FIG. 12, d, e), including a database of immunoglobulin germline-encoded variable region N-glycosylation sites³⁶. The absence of acetylated sialic acid on the Fc fragment and germline heavy and light chain variable regions suggested the functional impacts associated with anti-Lm IgG differential acetylation localized to novel N-glycan sites created by somatic hypermutation in the highly polymorphic Fab fragment³⁷.

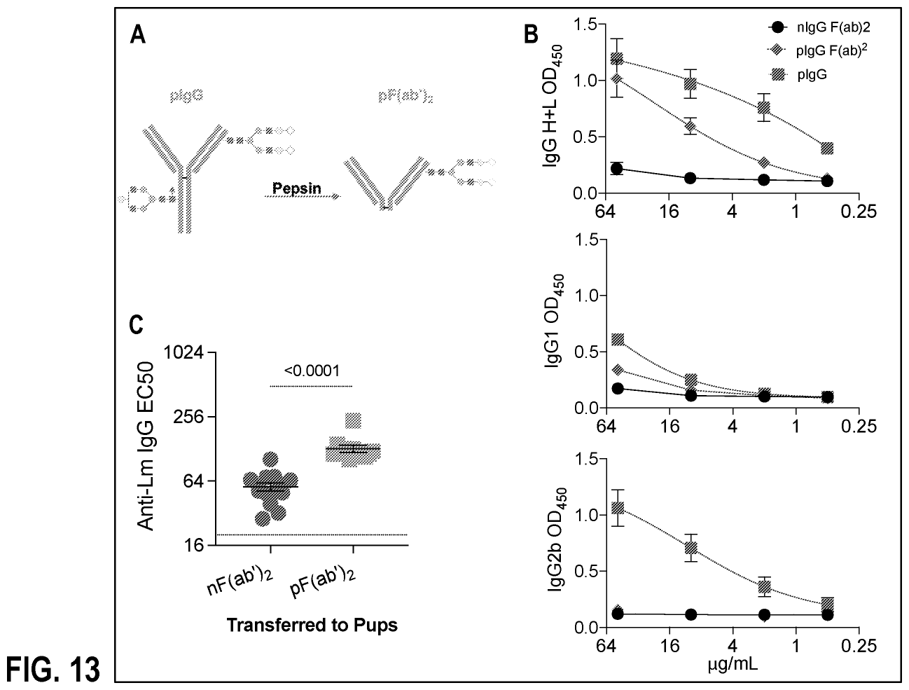

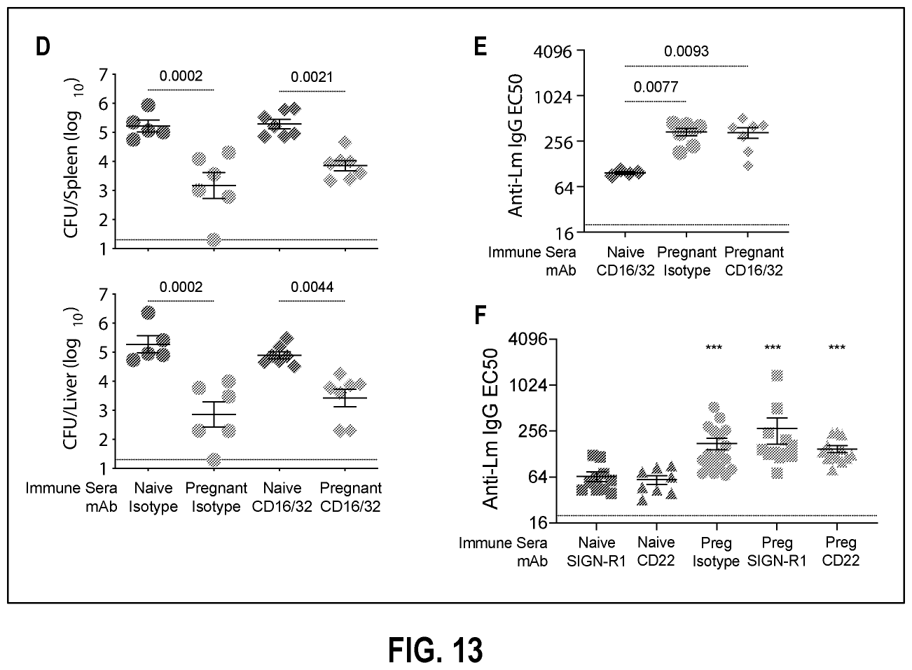

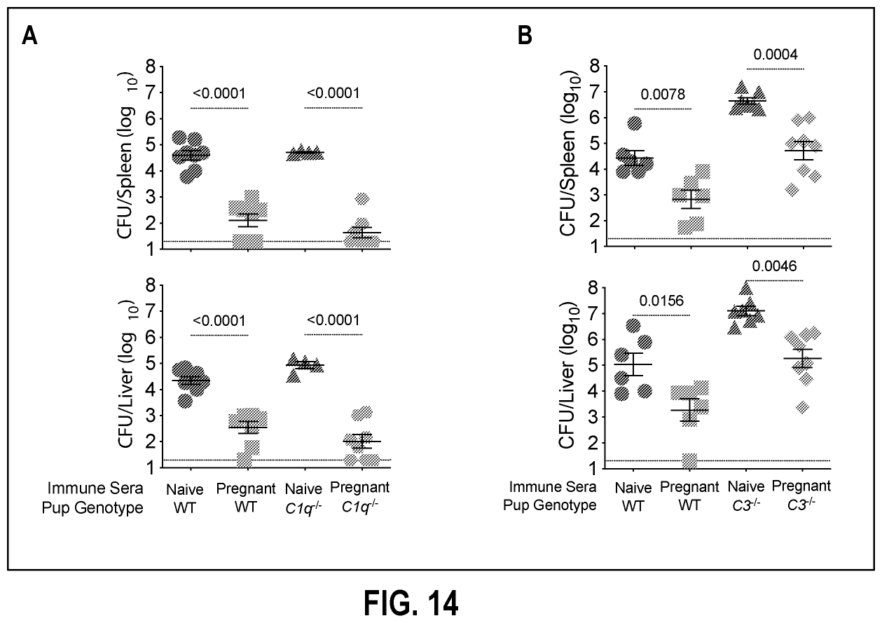

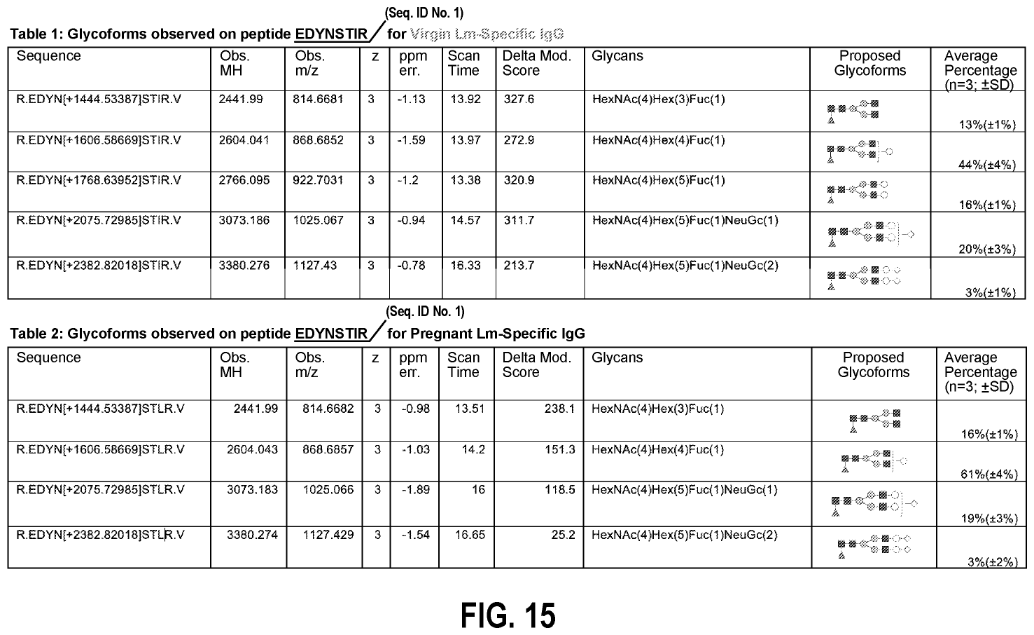

The unanticipated importance of IgG variable region N-glycan sialylation was confirmed by evaluating whether F(ab′)2 fragments alone confer protection. Sharply reduced bacterial burdens were found in neonatal mice passively transferred F(ab′)2 generated from anti-Lm IgG of pregnant mice (FIG. 4, f; FIG. 13, a-c). In agreement, Fc-mediated functions were dispensable, as anti-CD16/32 blocking antibody (FIG. 13, d,e) and complement deficiency in neonates (FIG. 14, a, b) each had no impact on protection against Lm infection mediated by adoptively transferred intact IgG. By contrast, the sialic acid binding receptors SIGN-R1 and CD22 were both essential, since neutralization of each in neonates overturned protection mediated by passively transferred anti-Lm antibodies from pregnant mice (FIG. 4, g; FIG. 13, f). Together, these results demonstrate that pregnancy expands antibody-mediated protection against intracellular infection via IgG variable region sialic acid deacetylation.

### Discussion

The foundational immunological tenet that humoral and cell-mediated adaptive immunity play non-overlapping functions is largely based on Lm infection experiments demonstrating that convalescent serum from mice with resolved infection cannot transfer protection to naïve recipient mice, whereas protection is readily transferred with donor splenocytes containing CD8+ T cells^{20,21,24,38,39}. However, these passive transfer studies exclusively using virgin adult animals have limited relevance to the unique susceptibility of newborn babies that rely on vertically transferred immunity. By considering the maternal-fetal dyad as a joined immunological unit, and the only known biological context where immune components are naturally transferred between individuals, unexpected protective roles for Lm-specific antibodies are revealed. Antibodies protect in the neonatal period when individuals are the most vulnerable in order to bypass susceptibility associated with immature T cell-mediated immunity. Protection by Lm-specific antibodies is not restricted to neonates, as protection was also observed for adult offspring born to Lm-primed mothers as well as Lm-primed CD8+ T cell-deficient mice after pregnancy.

A key distinction for Lm-primed antibodies is their ability to become activated through N-glycan sialic acid deacetylation. Protection in offspring occurred despite low-level vertical transfer of polyclonal maternal antibodies, therefore likely operating through different mechanisms from that previously shown for neutralizing anti-LLO monoclonal antibody clones that protected only at very high titer^{40,41}. These results fundamentally expand current knowledge regarding mechanisms of antibody-mediated protection against intracellular pathogens⁴², which have largely been thought to rely on extracellular neutralization or Fc receptor binding. For example, IgG transcytosis via the neonatal Fc receptor allowed intracellular influenza virus neutralization⁴³, while the intracytoplasmic Fc receptor TRIM21-binding to virus-specific antibodies promoted rotavirus proteasomal degradation⁴⁴. By contrast, pregnancy deacetylated anti-Lm IgG protects via sialic acid interaction with the C-type lectin SIGN-R145 (mouse homolog of human DC-SIGN) and the Siglec CD22.⁴⁶

Human SIAE polymorphisms have been linked with autoimmune disorders including rheumatoid arthritis and type I diabetes mellitus³⁵, while CD22 polymorphisms are associated with systemic lupus erythematosus⁴⁷. CD22 functionally represses B cell receptor signaling⁴⁸, and its sialylated ligands are silenced by acetylation⁴⁹. High affinity self-reactive antibody clones may be acetylated as a means to prevent autoimmunity, at the expense of expanded protection against intracellular infection. Sialic acid acetylation also reduces the activity of endogenous human neuraminidases⁵⁰, which may fine-tune antibody half-life and optimize embryogenesis since forced expression of sialic acid-deacetylating enzymes is associated with tissue-specific developmental defects in mice⁵¹. Placental SIAE expression also increases during pregnancy complications such as preeclampsia³⁴, suggesting that modulation of sialic acid acetylation may promote fetal tolerance. Acetylated sialic acid is also the target for several viral attachment proteins (e.g. influenza virus C, bovine and porcine coronaviruses)⁵², and therefore SIAE upregulation during pregnancy may decrease infection susceptibility.

Persistent susceptibility of neonates to intracellular infections, despite the potential for pregnancy-induced antibody modifications, may reflect inadequate maternal pathogen exposure, especially considering serotype diversity of common intracellular pathogens. For example, the risk of congenital cytomegalovirus is ˜20-fold increased for women with primary infection compared with secondary infection during pregnancy⁵³, while the risk of symptomatic congenital infection is especially increased by maternal reinfection with a serologically distinct CMV strain⁵⁴. Similarly, HSV resistance in neonates is associated with maternal type-specific antibodies⁵⁵. For Lm, commensal-pathobiont primed serological response for an estimated 5% fecal carriage in healthy adults¹⁵, together with expanded range protection by Lm-specific antibodies now demonstrated, likely explains the disproportionately small incidence of neonatal infection. In the broader biological context, consideration of the maternal-fetal dyad as a joined immunological unit unveils newfound protective roles for antibodies against intracellular infection, revising the foundational tenet that humoral and cell-mediated adaptive immune components have nonoverlapping functions. In turn, vertically transferred protection against intracellular infection mediated by antibody variable region sialic acid deacetylation reveals precise fine-tuning of host defenses to mitigate vulnerability during pregnancy and in early life.

### Methods

### Mice

Inbred C57BL/6 mice were purchased from the National Cancer Institute or generated by in-house breeding of CD45.1 or CD45.2 congenic mice on the C57BL/6 background. μMT−/−, CD8−/−, FcRγ−/−, C1q−/− and C3−/− were purchased from Jackson Laboratories. Mice were maintained under specific-pathogen-free conditions at Cincinnati Children's Hospital. Females between 8 and 12 weeks of age were used for all experiments. For some experiments, timed mating was performed by synchronized introduction of males to breeding cages. For experiments examining immunodeficient maternal mice, females were mated with WT males to generate heterozygous immunocompetent offspring. Mice were checked daily for birth timing. Neonates from the same litter were divided amongst groups for individual experiments. For cross foster experiments, pups were switched between nursing dams within 12 hours of birth. For survival experiments, mice were sacrificed when moribund. Adult mice were sacrificed via cervical dislocation. Neonates were sacrificed via decapitation. Experiments involving animals were performed under Cincinnati Children's Hospital Institutional Animal Care and Use Committee (IACUC) approved protocols.

### Bacteria and Infections

Listeria monocytogenes (wildtype strain 10403S or mutant DP-L1942) was grown in brain heart infusion (BHI) medium at 37° C., back diluted to early logarithmic phase (an optical density at 600 nm of 0.1) and resuspended in sterile PBS. DP-L1942 contains an in-frame deletion in the ActA gene (referred to as ΔActA Lm in the text). Female mice were preconceptually primed with ΔActA Lm 107 CFU injected intravenously (i.v.). For some experiments, a second injection was given 2 weeks later. Mice were mated 5-7 days after priming. For virulent infection, adult mice were inoculated i.v. with a dose of 2×10⁴ CFU per mouse, except FIG. 1d where mice received 105 CFU. Neonatal mice were infected with 50-100 CFU intraperitoneally (i.p.). For fungal infections, neonatal mice were infected i.p. with 106 CFU of Candida albicans grown in YPAD media and back diluted similarly to Lm. The inoculum for each experiment was verified by spreading a diluted aliquot onto agar plates. To assess susceptibility after infection, mouse organs (spleen and liver) were dissected and homogenized in sterile PBS containing 0.05% Triton X-100 to disperse the intracellular bacteria, and serial dilutions of the organ homogenate were spread onto agar plates. Colonies were counted after plate incubation at 37° C.

### Serum Harvest and IgG Purification

For phlebotomy, adult mice were bled 200 μL via submandibular bleed or via cardiac puncture at the time of sacrifice. For neonates, blood was collected after decapitation. To harvest serum, the blood was allowed to clot at room temperature and then spun at 10,000 rpm for 10 min. Serum was removed and then heat inactivated (56° C. for 20 min). Sera were collected separately and pooled from several Lm-primed mice. Immune sera from virgin mice (vSera) were collected starting 3 weeks after the last dose of ΔActA Lm. Immune sera from pregnant mice (pSera) were collected starting from late in gestation (˜E18) to post-partum day 5 (P5). Adoptive sera transfers were accomplished via i.p. injection in adults (200 μL volume) or neonates (50 μL volume). For breastmilk transfer of antibodies, nursing dams were injected with Lm-containing antibody sera from virgin mice when pups were P0 and P3 or P3 only (similar results were obtained).

Sera containing anti-Lm antibodies were purified over Protein A columns per manufacturer instructions (Abcam, catalog no. 109209) to obtain the IgG-containing fraction. Purified IgG was concentrated and dialyzed to PBS using the Amicon® Ultra Pro Purification System (Millipore, ACS510024). Protein G spin columns (Thermo, 89953) were utilized to isolate IgG from individual mice. IgG from naïve mice was purified from sera or purchased (Sigma, I5381). Neonates were transferred 50 □g purified IgG.

### Enzyme-Linked Immunosorbent Assays

For evaluating Lm-specific antibodies by enzyme-linked immunosorbent assay (ELISA), flat-bottom, high-binding, 96-well enzyme immunoassay (EIA)/radioimmunoassay (RIA) plates (Costar) were coated with nearly confluent log-phase Lm 10403S and allowed to dry overnight under UV light. Alternatively, plates were coated with recombinant LLO toxin at 1 μg/mL for at least 24 hours. Coated plates were then blocked with 3% milk. All wash steps were performed in triplicate with PBS+0.05% Tween-20. Serum from each mouse was diluted 1:10 or 1:20 and then 1:4 serial dilutions were performed followed by staining with the following biotin-conjugated anti-mouse secondary antibodies: rat anti-mouse IgG (eBioscience, 13-4013-8), rat anti-mouse IgM (eBioscience, 13-5890-1589), rat anti-mouse IgA (eBioscience, 13-5994-82), rat anti-mouse IgG1 (BD Pharmingen, cat. no. 553441), rat anti-mouse IgG2b (BD Pharmingen, 553393), rabbit anti-mouse IgG2c (Invitrogen, SA5-10235), and rat anti-mouse IgG3 (BD Pharmingen, 553401). Each secondary antibody was used at 1:1,000 dilution. Plates were developed with streptavidin-peroxidase (BD Bioscience, 554066) using o-phenylenediamine dihydrochloride as a substrate. Absorbance at 450 nm (A450) was read as described previously56. Antibody titers were quantified as EC50 (the point of 50% maximum OD450) using a nonlinear second order polynomial in Prism (GraphPad).

To detect N-glycans, LLO-coated plates were utilized to avoid staining endogenous glycans present on Lm bacteria. Plates were blocked with a carbohydrate-free blocking buffer (VectorLabs, SP-5040-125). Purified IgG was added at 0.1 mg/mL final concentration and then biotinylated lectins (all from VectorLabs) with defined carbohydrate specificity were used as secondary probes: SNA (terminal α2,6 Sialic Acid, 8 μg/mL concentration), ECA (β1,4 Galactose, 20 μg/mL concentration), AAL (α1,6 Fucose-β1,N-GlcNAc, 20 μg/mL concentration), UEA (α1,2 Fucose, 20 μg/mL concentration), and GSL-II (terminal GlcNAc, 20 μg/mL concentration). The biotinylated CCA lectin (EY Labs, BA-7201-1) with specificity for 9-O-acetylated sialic acid was used at 10 μg/mL concentration and plates were developed using SuperSignal ELISA Femto Maximum Sensitivity Substrate (Thermo, 37075) and lumens were detected using the Synergy Neo2 plate reader (BioTek).

### IgG Enzyme Treatments

In vivo IgG deglycosylation in nursing dams that had been transferred sera containing Lm-specific IgG was accomplished by i.v. injection with 10 □g of recombinant low-endotoxin EndoS2 (GlycINATOR LE, Genovis, A0-GL8-020) diluted in sterile PBS29. Purified IgG was digested with EndoS (NEB, P0741L, 1 μL per 100 μg IgG, pH 5.5) to remove N-linked glycans from the heavy chain of native IgG. Neuraminidase (NEB, P0720L, 1 μL per 25 μg IgG, pH 5.5) was used to remove sialic acid and then the enzyme was functionally inactivated by incubation at 55° C. for 10 min. IgG was then separated from neuraminidase by size exclusion chromatography (100 kDa MWCO) before being resialylated using ST6Gal1 (Creative Biomart, St6gal1-7036M, 1 μg per 20 μg IgG, pH 7.0) plus 2.5 mM CMP-acetylneuraminic acid (CMP-Neu5Ac, Calbiochem, 5052230001) as substrate. IgG N-glycan sialic acid deacetylation was accomplished by treating IgG with sialic acid acetyl esterase (SIAE, Creative Biomart, SIAE-15119M, 1 μL per 40 μg IgG, pH 8.0). All glycosylation modifying enzyme reactions were performed at 37° C. for 20-24 hours and success was confirmed by lectin staining. To generate F(ab′)2 fragments, IgG was treated with pepsin (Thermo, 44988) at pH 4, 37° C. for 4-5 hours and purified by size exclusion chromatography (50 kDa MWCO) then dialyzed to PBS. Successful cleavage of Fc was confirmed via gel analysis and ELISA with IgG subtype specific secondary antibodies.

### Lm-Specific Antibody Isolation

Serum from virgin or pregnant mice was buffer exchanged into 20 mM NaH₂PO₄ pH 7.0, 150 mM NaCl using a HiPrep 26/10 Desalting 53 mL column (Cytiva) and run over a HiTrap Protein G HP 1 mL column (Cytiva) to capture antibodies. Antibodies were eluted with 100 mM glycine pH ˜2 into tubes containing sufficient Tris to neutralize the pH. UV-inactivated Lm (UV Stratalinker 2400, Stratagene, 6 min total treatment) was centrifuged at 4,000 rpm for 5 minutes to pellet the bacteria. Then, Lm pellets were resuspended in 20 mM NaH₂PO₄ pH 7.0, 150 mM NaCl. The pellets were washed by centrifuging for another 5 minutes, pouring off the supernatant, then resuspending with fresh buffer. Bacteria were washed a total of 3 times, then the final pellet from 2-8 L of Lm was resuspended with 2.5 mL of virgin- or pregnant-derived, Protein G-purified antibodies at ˜0.1-0.2 mg/ml. This mixture was incubated for 30 minutes with gentle shaking at room temperature. The bacteria were again centrifuged for 5 min, and the supernatant was discarded. The pellet containing Lm and Lm-specific antibodies, was washed with 10 mL of buffer by resuspension, then centrifuged for 5 minutes. The supernatant was discarded before adding 2.5 mL of buffer containing 2 M MgCl2 to elute the Lm-specific antibodies from the bacteria. The mixture was incubated with shaking for another 5 minutes and centrifuged for 5 minutes. The supernatant containing Lm-specific antibodies was collected and buffer-exchanged using a HiPrep 26/10 Desalting 53 mL column (Cytiva) to remove the MgCl2. The antibodies were again purified using a HiTrap Protein G HP 1 mL column (Cytiva), then buffer exchanged back into 20 mM NaH2PO4 pH 7.0, 150 mM NaCl during concentration of the antibodies for downstream applications.

### Mass Spectroscopy

Water (Honeywell), acetonitrile (ACN; Fisher) and formic acid (FA; Sigma) were all of LC-MS grade. All other chemicals were of laboratory analytical reagent grade. Samples were reduced, alkylated, then digested into peptides. A solution containing ˜20 μg of the protein solution in 50 mM Tris-Cl (pH 7.4) was reduced in a solution of 5 mM dithiothreitol (DTT) at 45° C. for 45 minutes, a solution of iodoacetamide (IAA) was added to bring the solution to 15 mM and then incubated at room temperature in the dark for 45 minutes. A second aliquot of DTT was then added to the solution to quench the remaining IAA. Trypsin or Chymotrypsin (Promega, Sequencing Grade) was added to the solution and allowed to digest for 16 hours. The digestion was stopped by briefly heating the solution to 100° C. for 5 minutes before cooling. The digested material was then injected for LC-MS.

LC-MS/MS was performed on an Orbitrap Eclipse Tribrid MS (Thermo Fisher Scientific, Massachusetts, USA) coupled to a Ultimate RSLCnano 3000 (Thermo Fisher Scientific, Massachusetts, USA) and equipped with an nanospray ion source. Prepared samples were injected to the separation column (Acclaim PepMap 100 75 μm×15 cm). The separation was performed in a linear gradient from low to high acetonitrile containing 0.1% formic acid. Mass spectrometry was carried out in the positive ion mode where a full MS spectrum was collected at high resolution (120,000) and data dependent MS/MS scans of the highest intensity peaks following HCD fragmentation were collected in the Orbitrap. HCD fragments corresponding to sialic acid oxonium ions were then subsequently fragmented a second time with ETD fragmentation. The LC-MS/MS data were analyzed using Byonic (version 4.0) software search and glycopeptide annotations were screened manually for b and y ions, glycan oxonium ion, and neutral losses. Quantification of peak intensities were calculated manually with the instrument software (Xcalibur, 4.2) based on deconvoluted spectra. Manual sorting of MS/MS fragmentation to search for oxonium ions consistent with acetyl-glycolylneuraminic acid (Neu5Gc,Ac) was also conducted with the instrument software using a 5 ppm range of mass error, which is consistent with MS/MS data collected in the Orbitrap for this instrument.

RNA Isolation and qPCR

For mice, spleen RNA was extracted from virgin or pregnant females at late gestation (E18-20). For humans, peripheral blood was collected from de-identified non-pregnant or pregnant adult volunteers from any time during gestation up until delivery under Cincinnati Children's Hospital Medical Center institutional review board (IRB) approved protocols (IRB ID 2020-0991). Mononuclear cells were freshly isolated over Ficoll-Hypaque gradients. Frozen buffy coats from non-pregnant and pregnant patients were thawed and total RNA isolated using the RNAaqueous-4PCR kit (Invitrogen, AM1914). cDNA synthesis was performed using the TaqMan Reverse Transcription kit (Applied Biosystems, N808234) with an Oligo-d(T)16 nucleotide reverse transcription primer. qPCR reactions were set up using the Taq Man Fast Advanced Master Mix (Applied Biosystems, 4444556). qPCR was performed on a 7500 Fast Real-Time PCR System (Applied Biosystems) using exon-spanning TaqMan probes (Thermo Fischer) for mouse β-Actin (Mm04394036_g1), mouse SIAE (Mm00496036_m1), human RPL13A (Hs03043885_g1), or human SIAE (Hs00405149_m1). SIAE gene expression was normalized to the housekeeping gene and fold increase calculated using the 2^{ΔΔCt} method.

### In Vivo Antibody Blockade

To block antibody receptors in vivo, 3 d old mouse pups were injected with one of the following blocking mAb or appropriate isotype control: anti-mouse CD16/CD32 (Bio Xcell, BE0307, 100 μg/pup), anti-mouse SIGN-R1 (BioXcell, BE0220, 50 μg/pup), anti-mouse CD22 (BioXcell, BE0011, 100 μg/pup). Blocking antibodies were given simultaneously with anti-Lm antibodies. Neonatal mice were infected with Lm the following day.

### Quantification and Statistical Analysis

The number of individual animals used per group are described in each individual figure panel or shown by individual data points that represent the results from individual animals. Statistical tests were performed using Prism (GraphPad) software. The unpaired two-tailed Student's t test was used to compared differences between two groups. One-way ANOVA with Bonferroni post-test for multiple comparisons was used to evaluate experiments containing more than two groups. Limits of detection for each assay are denoted by a dotted horizontal line.

### REFERENCES

1. Collins F M. Cellular antimicrobial immunity. CRC Crit Rev Microbiol. 1978; 7(1):27-91. doi:10.3109/10408417909101177
2. Mackaness G B. Resistance to intracellular infection. J Infect Dis. April 1971; 123(4):439-45. doi:10.1093/infdis/123.4.439
3. Albrecht M, Arck P C. Vertically Transferred Immunity in Neonates: Mothers, Mechanisms and Mediators. Front Immunol. 2020; 11:555. doi:10.3389/fimmu.2020.00555
4. Visser E A, Moons S J, Timmermans SBPE, de Jong H, Boltje T J, Bull C. Sialic acid O-acetylation: From biosynthesis to roles in health and disease. J Biol Chem. June 2021:100906. doi:10.1016/j.jbc.2021.100906
5. Bochner B S, Zimmermann N. Role of siglecs and related glycan-binding proteins in immune responses and immunoregulation. J Allergy Clin Immunol. March 2015; 135(3):598-608. doi:10.1016/j.jaci.2014.11.031
6. Meyer S J, Linder A T, Brandl C, Nitschke L. B Cell Siglecs-News on Signaling and Its Interplay With Ligand Binding. Front Immunol. 2018; 9:2820. doi:10.3389/fimmu.2018.02820
7. Wang H, Liddell C A, Coates M M, et al. Global, regional, and national levels of neonatal, infant, and under-5 mortality during 1990-2013: a systematic analysis for the Global Burden of Disease Study 2013. Lancet. September 2014; 384(9947):957-79. doi:10.1016/S0140-6736(14)60497-9
8. Wardlaw T, You D, Hug L, Amouzou A, Newby H. UNICEF Report: enormous progress in child survival but greater focus on newborns urgently needed. Reprod Health. December 2014; 11:82. doi:10.1186/1742-4755-11-82
9. Robbins J R, Bakardjiev A I. Pathogens and the placental fortress. Curr Opin Microbiol. February 2012; 15(1):36-43. doi:10.1016/j.mib.2011.11.006
10. Posfay-Barbe K M, Wald E R. Listeriosis. Semin Fetal Neonatal Med. August 2009; 14(4):228-33. doi:10.1016/j.siny.2009.01.006
11. Radoshevich L, Cossart P. Listeria monocytogenes: towards a complete picture of its physiology and pathogenesis. Nat Rev Microbiol. January 2018; 16(1):32-46. doi:10.1038/nrmicro.2017.126
12. Portnoy D A, Auerbuch V, Glomski I J. The cell biology of Listeria monocytogenes infection: the intersection of bacterial pathogenesis and cell-mediated immunity. J Cell Biol. August 2002; 158(3):409-14. doi:10.1083/jcb.200205009
13. Marchant A, Sadarangani M, Garand M, et al. Maternal immunisation: collaborating with mother nature. Lancet Infect Dis. 07 2017; 17(7):e197-e208. doi:10.1016/S1473-3099(17)30229-3
14. Abu Raya B, Edwards K M, Scheifele D W, Halperin S A. Pertussis and influenza immunisation during pregnancy: a landscape review. Lancet Infect Dis. 07 2017; 17(7):e209-e222. doi:10.1016/51473-3099(17)30190-1
15. Zheng W, Zhao W, Wu M, et al. Microbiota-targeted maternal antibodies protect neonates from enteric infection. Nature. 01 2020; 577(7791):543-548. doi:10.1038/s41586-019-1898-4
16. Kollmann T R, Marchant A, Way S S. Vaccination strategies to enhance immunity in neonates. Science. 05 2020; 368(6491):612-615. doi:10.1126/science.aaz9447
17. Fouda G G, Martinez D R, Swamy G K, Permar S R. The Impact of IgG transplacental transfer on early life immunity. Immunohorizons. January 2018; 2(1):14-25. doi:10.4049/immunohorizons.1700057
18. Stuebe A. The risks of not breastfeeding for mothers and infants. Rev Obstet Gynecol. 2009; 2(4):222-31.
19. MACKANESS G B. Cellular resistance to infection. J Exp Med. September 1962; 116:381-406. doi:10.1084/jem.116.3.381
20. OSEBOLD J W, SAWYER M T. Immunization studies on listeriosis in mice. J Immunol. April 1957; 78(4):262-8.
21. Kaufmann S H, Hug E, De Libero G. Listeria monocytogenes-reactive T lymphocyte clones with cytolytic activity against infected target cells. J Exp Med. July 1986; 164(1):363-8. doi:10.1084/jem.164.1.363
22. Bishop D K, Hinrichs D J. Adoptive transfer of immunity to Listeria monocytogenes. The influence of in vitro stimulation on lymphocyte subset requirements. J Immunol. September 1987; 139(6):2005-9.
23. Mielke M E, Ehlers S, Hahn H. T-cell subsets in delayed-type hypersensitivity, protection, and granuloma formation in primary and secondary Listeria infection in mice: superior role of Lyt-2+ cells in acquired immunity. Infect Immun. August 1988; 56(8):1920-5. doi:10.1128/iai.56.8.1920-1925.1988
24. North R J. Cellular mediators of anti-Listeria immunity as an enlarged population of short lived, replicating T cells. Kinetics of their production. J Exp Med. August 1973; 138(2):342-55. doi:10.1084/jem.138.2.342
25. Harty J T, Bevan M J. Specific immunity to Listeria monocytogenes in the absence of IFN gamma. Immunity. July 1995; 3(1):109-17. doi:10.1016/1074-7613(95)90163-9
26. Bruhns P, Jönsson F. Mouse and human FcR effector functions. Immunol Rev. November 2015; 268(1):25-51. doi:10.1111/imr.12350
27. Jennewein M F, Goldfarb I, Dolatshahi S, et al. Fc Glycan-Mediated Regulation of Placental Antibody Transfer. Cell. 06 2019; 178(1):202-215.e14. doi:10.1016/j.cell.2019.05.044
28. Jennewein M F, Alter G. The Immunoregulatory Roles of Antibody Glycosylation. Trends Immunol. 05 2017; 38(5):358-372. doi:10.1016/j.it.2017.02.004
29. Albert H, Collin M, Dudziak D, Ravetch J V, Nimmerjahn F. In vivo enzymatic modulation of IgG glycosylation inhibits autoimmune disease in an IgG subclass-dependent manner. Proc Natl Acad Sci USA. September 2008; 105(39):15005-9. doi:10.1073/pnas.0808248105
30. Traving C, Schauer R. Structure, function and metabolism of sialic acids. Cell Mol Life Sci. December 1998; 54(12):1330-49. doi:10.1007/s000180050258
31. Medzihradszky K F, Kaasik K, Chalkley R J. Characterizing sialic acid variants at the glycopeptide level. Anal Chem. March 2015; 87(5):3064-71. doi:10.1021/ac504725r
32. Ravindranath M H, Higa H H, Cooper E L, Paulson J C. Purification and characterization of an O-acetylsialic acid-specific lectin from a marine crab Cancer antennarius. J Biol Chem. July 1985; 260(15):8850-6.
33. Cariappa A, Takematsu H, Liu H, et al. B cell antigen receptor signal strength and peripheral B cell development are regulated by a 9-O-acetyl sialic acid esterase. J Exp Med. January 2009; 206(1):125-38. doi:10.1084/jem.20081399
34. Tsai S, Hardison N E, James A H, et al. Transcriptional profiling of human placentas from pregnancies complicated by preeclampsia reveals disregulation of sialic acid acetylesterase and immune signalling pathways. Placenta. February 2011; 32(2):175-82. doi:10.1016/j.placenta.2010.11.014
35. Surolia I, Pirnie S P, Chellappa V, et al. Functionally defective germline variants of sialic acid acetylesterase in autoimmunity. Nature. July 2010; 466(7303):243-7. doi:10.1038/nature09115
36. Melo-Braga M N, Carvalho M B, Emiliano M C, Ferreira, Felicori L F. New insights of glycosylation role on variable domain of antibody structures. bioRxiv2021.
37. van de Bovenkamp F S, Hafkenscheid L, Rispens T, Rombouts Y. The Emerging Importance of IgG Fab Glycosylation in Immunity. J Immunol. February 2016; 196(4):1435-41. doi:10.4049/jimmunol.1502136
38. North R J, Dunn P L, Conlan J W. Murine listeriosis as a model of antimicrobial defense. Immunol Rev. August 1997; 158:27-36. doi:10.1111/j.1600-065x.1997.tb00989.x
39. Unanue E R. Studies in listeriosis show the strong symbiosis between the innate cellular system and the T-cell response. Immunol Rev. August 1997; 158:11-25. doi:10.1111/j.1600-065x.1997.tb00988.x
40. Edelson B T, Cossart P, Unanue E R. Cutting edge: paradigm revisited: antibody provides resistance to Listeria infection. J Immunol. October 1999; 163(8):4087-90.
41. Edelson B T, Unanue E R. Intracellular antibody neutralizes Listeria growth. Immunity. May 2001; 14(5):503-12. doi:10.1016/s1074-7613(01)00139-x
42. Casadevall A, Dadachova E, Pirofski L A. Passive antibody therapy for infectious diseases. Nat Rev Microbiol. September 2004; 2(9):695-703. doi:10.1038/nrmicro974
43. Bai Y, Ye L, Tesar D B, et al. Intracellular neutralization of viral infection in polarized epithelial cells by neonatal Fc receptor (FcRn)-mediated IgG transport. Proc Natl Acad Sci USA. November 2011; 108(45):18406-11. doi:10.1073/pnas.1115348108
44. Caddy S L, Vaysburd M, Wing M, et al. Intracellular neutralisation of rotavirus by VP6-specific IgG. PLoS Pathog. 08 2020; 16(8):e1008732. doi:10.1371/journal.ppat.1008732
45. Massoud A H, Yona M, Xue D, et al. Dendritic cell immunoreceptor: a novel receptor for intravenous immunoglobulin mediates induction of regulatory T cells. J Allergy Clin Immunol. March 2014; 133(3):853-63.e5. doi:10.1016/j.jaci.2013.09.029
46. Séïté J F, Cornec D, Renaudineau Y, Youinou P, Mageed R A, Hillion S. IVIg modulates BCR signaling through CD22 and promotes apoptosis in mature human B lymphocytes. Blood. September 2010; 116(10):1698-704. doi:10.1182/blood-2009-12-261461
47. Hatta Y, Tsuchiya N, Matsushita M, Shiota M, Hagiwara K, Tokunaga K. Identification of the gene variations in human CD22. Immunogenetics. April 1999; 49(4):280-6. doi:10.1007/s002510050494
48. Clark E A, Giltiay N V. CD22: A Regulator of Innate and Adaptive B Cell Responses and Autoimmunity. Front Immunol. 2018; 9:2235. doi:10.3389/fimmu.2018.02235
49. Sjoberg E R, Powell L D, Klein A, Varki A. Natural ligands of the B cell adhesion molecule CD22 beta can be masked by 9-O-acetylation of sialic acids. J Cell Biol. July 1994; 126(2):549-62. doi:10.1083/jcb.126.2.549
50. Hunter C D, Khanna N, Richards M R, et al. Human Neuraminidase Isoenzymes Show Variable Activities for 9-O-Acetyl-sialoside Substrates. ACS Chem Biol. 04 2018; 13(4):922-932. doi:10.1021/acschembio.7b00952
51. Varki A, Hooshmand F, Diaz S, Varki N M, Hedrick S M. Developmental abnormalities in transgenic mice expressing a sialic acid-specific 9-O-acetylesterase. Cell. April 1991; 65(1):65-74. doi:10.1016/0092-8674(91)90408-q
52. Langereis M A, Bakkers M J, Deng L, et al. Complexity and Diversity of the Mammalian Sialome Revealed by Nidovirus Virolectins. Cell Rep. June 2015; 11(12):1966-78. doi:10.1016/j.celrep.2015.05.044
53. Fowler K B, Stagno S, Pass R F, Britt W J, Boll T J, Alford C A. The outcome of congenital cytomegalovirus infection in relation to maternal antibody status. N Engl J Med. March 1992; 326(10):663-7. doi:10.1056/NEJM199203053261003
54. Boppana S B, Rivera L B, Fowler K B, Mach M, Britt W J. Intrauterine transmission of cytomegalovirus to infants of women with preconceptional immunity. N Engl J Med. May 2001; 344(18):1366-71. doi:10.1056/NEJM200105033441804
55. Brown Z A, Wald A, Morrow R A, Selke S, Zeh J, Corey L. Effect of serologic status and cesarean delivery on transmission rates of herpes simplex virus from mother to infant. JAMA. January 2003; 289(2):203-9. doi:10.1001/jama.289.2.203
56. Turner L H, Kinder J M, Wilburn A, et al. Preconceptual Zika virus asymptomatic infection protects against secondary prenatal infection. PLoS Pathog. November 2017; 13(11):e1006684. doi:10.1371/joumal.ppat.1006684

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of modulating antibody function, comprising modifying a terminal sialic acid residue of said antibody.

2. The method of claim 1, wherein said modification comprises removing an existing sialic acid, and replacing said sialic acid with a sialic acid variant of interest.

3. The method of claim 2, comprising treatment with neuraminidase, followed by treatment with ST6Gal1 and the sialic acid variant of interest.

4. The method of claim 1, wherein said sialic acid is selected from N-glycolylneuraminic acid (NGNA or Neu5Gc), where a N-acetyl group of Neu5Ac is hydroxylated, 2-keto-3-deoxynonurosonic acid (KDN), a 9-substituted sialic acid, 9¬O—C—C6 acyl Neu5Ac, a 9-deoxy, 9-O-lactyl-Neu5Ac, 9-O-acetyl-Neu5Ac), 4-O-Acetyl-SA, 7,9-O-Acetyl-SA, 9-Fluoro-Neu5Ac, 9Azido-9-deoxy-Neu5Ac, and combinations thereof.

5. (canceled)

6. The method of claim 1 wherein said antibody function is one or more of a protective effect, a therapeutic effect, and an immune response to an antigen.

7. The method of claim 1 wherein said modulating comprises an increase or decrease in said antibody function.

8. The method of claim 1 wherein said modifying comprises removing a terminal sialic acid residue of said antibody.

9. The method of claim 1 wherein said modifying comprises adding a terminal sialic acid residue of said antibody.

10. The method of claim 1 wherein said modifying is acetylation of said terminal sialic acid residue.

11. The method of claim 1 wherein said modifying is de-acetylation of said terminal sialic acid residue.

12. The method of claim 11 wherein said de-acetylation is carried out via contact with sialic acid acetyl esterase (SIAE).

13. The method of claim 1, comprising contacting said antibody with ST6 beta-galactosidase alpha-2,6-sialyltransferase 1 (ST6Gal1) sufficient to add at least one α2,6-linked sialic acid to a galactose residue of said antibody.

14. The method of claim 1, comprising contacting said antibody with an endoglycosidase.

15. The method of claim 1, comprising contacting said antibody with neuraminidase digestion until said antibody is desialylated.

16. The method of claim 1, comprising contacting said antibody with neuraminidase followed by treatment with a sialyltransferase.

17. The method of claim 1, comprising contacting said antibody with neuraminidase followed by treatment with a sialyltransferase selected from ST3Gal III (α-(2,3) sialyltransferase and α-(2,6) sialyltransferase.

18. The method of claim 1, comprising contacting said antibody with one or more of ST6 beta-galactosidase alpha-2,6-sialyltransferase 1 (ST6Gal1) enzyme and a sialic acid.

19. The method of claim 18 wherein said sialic acid is acetylneuraminic acid (Neu5Ac).

20. The method of claim 18, wherein said sialic acid derivative is added to an IgG, said sialic acid being directly acetylated.

21. The method of claim 1 wherein said antibody is an immunoglobulin G-type (IgG) antibody.

22. The method of claim 1, wherein said antibody is an immunoglobulin G-type (IgG) antibody, and wherein said terminal sialic residue is located on a heavy chain of said IgG antibody.

23. The method of claim 21, wherein said terminal sialic acid residue is located on a variable region of a heavy chain of said IgG antibody.

24. The method of claim 23, wherein said terminal sialic acid residue is located on an N-linked glycan in a IgG variable region of said antibody.

25. (canceled)

26. (canceled)

27. A method of modulating an antibody-related immune response in an individual, comprising administering an antibody according to claim 1.

28. The method of claim 27, comprising administering said antibody in an amount and for a period of time sufficient to improve a disease outcome.

29. The method of claim 28, said outcome being selected from reduction in pathogen burden and survival, disease-free survival, symptom improvement or survival, and combinations thereof.

30. A composition comprising an antibody according to claim 1 and a pharmaceutically acceptable excipient.

31. (canceled)

32. (canceled)


SEQUENCE LISTING (3 sequences)

SEQ ID NO: 1 - protein, 8 aa, Artificial Sequence
  Glycopeptide
EDYNSTLR

SEQ ID NO: 2 - protein, 11 aa, Artificial Sequence
  Glycopeptide
NEESLYTAAIK

SEQ ID NO: 3 - protein, 13 aa, Artificial Sequence
  Glycopeptide
ANGGTTTPIQVDS